(19) United States
(12) Patent Application Publication
(10) Pub. No.: US 2014/0142060 A1
(43) Pub. Date: May 22, 2014

(54) METHOD AND DEVICE FOR IDENTIFICATION OF ONE CARBON PATHWAY GENE VARIANTS AS STROKE RISK MARKERS, COMBINED DATA MINING, LOGISTIC REGRESSION, AND PATHWAY ANALYSIS

(71) Applicants: Stuart A. Stein, Santa Ana, CA (US); Reed M. Stein, Houston, TX (US)

(72) Inventors: Stuart A. Stein, Santa Ana, CA (US); Reed M. Stein, Houston, TX (US)

(73) Assignee: Hyde Park West, LLC, Tucson, AZ (US)

(21) Appl. No.: 14/084,039

(22) Filed: Nov. 19, 2013

Related U.S. Application Data
(60) Provisional application No. 61/728,797, filed on Nov. 20, 2012.

(51) Int. Cl. C12Q 1/68; A61K 31/7056

(52) U.S. Cl. CPC C12Q 1/6883; A61K 31/7056
USPC 514/52; 435/6.11; 435/287.2

(57) ABSTRACT

The disclosure provides method and device using a whole genome association analysis toolset for a total stroke case, versus a control group, which demonstrated significant associations with p less than 1.00E.03 for 5 genes, including polymorphisms in MTHFR, MTRR, and BHMT. These gene polymorphisms in MTHFR may remove or create intron and exon splice enhancer sites that affect alternative splicing activity as well as appropriate mRNA and protein production.

### CROSS REFERENCE TO RELATED APPLICATION

This application claims the full Paris Convention priority to and benefit of U.S. Provisional Application Ser. No. 61/728,797, filed on Nov. 20, 2012, and entitled, “Method and Device for Identification of One Carbon Pathway Gene Variants as Stroke Risk Markers: Combined Data Mining, Logistic Regression, and Pathway Analysis,” the contents of which are hereby incorporated by this reference, as if fully set forth herein in their entirety.

### FIELD OF THE DISCLOSURE

The present disclosure relates to the detection of risk factors that predict early stroke. The risk factors take the form of Single Nucleotide Polymorphisms (SNPs) in the genomic DNA of human subjects. The disclosure uses an algorithm that correlates the existence of one or more particular SNPs in a human subject, and risk for early stroke, and provides guidance as to whether the human subject is or is not at increased risk for early stroke. The present disclosure uses an available database, focuses on genes that mediate 1-carbon metabolism, and using an algorithm, reviews the hundreds of SNPs that exist in these genes, and identifies a relatively small list of SNPs that predict increased risk for early stroke.

### BACKGROUND OF THE DISCLOSURE

Stroke is the third leading cause of death in the United States. Stroke is an abrupt interruption of constant blood flow to the brain that causes loss of neurological function. The most common type of stroke is ischemic stroke, and the second most common type of stroke is intracerebral hemorrhage (Ikram et al (2012) Curr. Atheroscler. Rep. 14:300-306). In the United States, 87% of all strokes are ischemic strokes, and 13% are hemorrhagic strokes, where these hemorrhagic strokes occur in intracerebral or subarachnoid locations (Yew and Cheng (2009) Am. Family Physician. 80:33-40). Ischemic stroke can take the form of cerebral thrombosis, where a blood clot forms inside a diseased or damaged artery in the brain resulting from atherosclerotic plaque. Ischemic stroke can also take the form of cerebral embolism, which occurs when a clot or a piece of atherosclerotic plaque travels through the bloodstream and lodges in narrower brain arteries. Hemorrhagic stroke can take the form of subarachnoid hemorrhage, which is bleeding that occurs in the space between the surface of them brain and the skull. Causes of subarachnoid hemorrhagic stroke are ruptured cerebral aneurysm, an area where a blood vessel in the brain weakens, resulting in a bulging or ballooning out of part of the vessel wall, and rupture of an arteriovenous malformation, a tangle of abnormal and poorly formed blood vessels with an innate propensity to bleed. Hemorrhagic stroke also takes the form of intracerebral hemorrhage, where bleeding occurs within the brain tissue. Intracerebral hemorrhages are due to changes in the arteries caused by long-term hypertension (American Association of Neurological Surgeons (AANS.org) (March 2011) Patient Information. Stroke).

The risk of death varies across stroke types, with lower mortality occurring with ischemic stroke, and higher mortality occurring with intracerebral hemorrhage and subarachnoid hemorrhage (Smith et al (2013) 2:e005207 (10 pages). Intracerebral hemorrhage (IH) occurs when a blood vessel within the brain parenchyma ruptures (Ikram et al (2012) Curr. Atheroscler. Rep. 14:300-306). Intracerebral hemorrhage is divided into spontaneous (non-lesional) ICH, which is parenchymal hemorrhage that occurs in the absence of underlying lesion, and secondary ICH, which occurs in the setting of a lesion such as a cerebral tumor or a vascular malformation (Sussman and Connolly (2013) Frontiers Neurology. 4 (7 pages)).

Regarding early onset stroke, the prevalence of the various classes of ischemic stroke and of intracerebral hemorrhage has been documented in young adults. For example, a study of ischemic stroke in young adults reveals that large artery atherosclerosis (8%), cardiac embolism (48%), small vessel disease (18%), various other etiologies (e.g., cervical artery dissection, migrainous infarction, etc.) (61%), and undetermined etiology (68%), occurred at the indicated prevalence (Nedeltchev et al (2005) J. Neurol. Neurosurg. Psychiatry. 76:191-196). These particular classes of ischemic stroke are those set forth by the TOAST classification. Regarding etiology, a study of young adults revealed that the causes of ischemic stroke were, atherosclerotic vasculopathy (9%), nonatherosclerotic vasculopathy (4%), vasculopathy of uncertain etiology (lacunar infarct) (21%), cardioembolic/transcardiac embolism (20%), hematololgic etiologies (14%), recreational drug related (6%) (Qureshi et al (1995) Stroke. 26:1995-1998). Regarding the meaning of “infarction,” infarction in the majority of cases refers to ischemic stroke. Hemorrhage can also be associated with infarction as part of the hemorrhage damage.

About 3-4% of cerebral ischemic infarctions occur in younger adults and in adolescents (ages 15-45) (Kappelle et al (1994) Stroke. 25:1360-1365).

The present disclosure addresses an unmet need. Although hypertension and diabetes are well-established risk factors for ischemic stroke in older adults, these particular risk factors are not as relevant for stroke in younger adults (Ferro et al (2010) Lancet Neurol. 9:1085-1096). Your et al (1997) Stroke. 28:1913-1918, similarly have stated that, “major risk factors for cerebral infarction in young adults, surprisingly, have rarely been studies systematically.” Accordingly, the present disclosure fulfills an unmet need by identifying risk factors for stroke in younger adults, and by providing a system and methods. The system and methods of the present disclosure were based on a dataset used from an early onset ischemic stroke data set and does not include hemorrhagic stroke. Also, for the present system and method, there was no subtype differentiation for ischemic stroke in the dataset made available. The system and method of the present disclosure also provides an algorithm for use in selecting SNPs from a population of early onset stroke victims, where this algorithm is, “Early Age Onset Stroke Association Algorithm.” In applying the novel Early Age Onset Stroke Association Algorithm to the GENEVA dataset, the inventors arrived at a selected group of 65 SNPs that were significantly correlated with early onset stroke. These 65 SNPs included 12 with a p less than 0.01. With these 65 SNPs in hand, the inventors manually reviewed the P values, and selected the twelve SNPs with the lowest P values.

Regarding intracerebral hemorrhage in young adults, the most common cause has been reported to be vascular malformations (49%) and hypertension (11%) (Ruiz-Sandoval et al (1999) Stroke. 30:537-541).

The present disclosure fulfills an unmet need, by providing a system and method, for conducting genetic analysis on a human patient, determining the presence or absence of specific mutations in genes that mediate 1-carbon metabolism, where the presence or absence assigns a risk for early stroke, followed by subjecting the patient to diagnostic methods (e.g., ultrasound) or pharmaceutical agents (e.g., folic acid) that have a physical influence on the patient's body.

The present disclosure relates to using a whole genome association analysis toolset for a total stroke case, versus a control group, which demonstrated significant associations with p less than 1.00E.03 for 5 genes, including polymorphisms in MTHFR [rs4846052 (intronic), rs7533315 (intronic), rs4846051 (intronic), rs6541003 (exonic)], MTRR (rs1802059), TYMS (rs2847149, rs2244500, rs1001761), BHMT (rs6893970), and SCL19A3 (rs13007334). 65 polymorphisms within the one-carbon folate genes were identified with p values less than 1.00E.01. MTHFR rs4846052 and rs7533315 were significantly associated with sex. The specific nucleotide polymorphism in MTHFR rs4846052 and rs7533315 intronic SNPs lead to removal of consensus Fox 1 and 2, intron splice enhancer sites (ISE); the MTHFR exonic SNP rs6864051, which is contiguous to rs1801131, a pathological SNP (A1298C) does not produce a projected pathological SNP but the nucleotide change removes a consensus exon splice enhancer site (ESS). These gene polymorphisms in MTHFR may remove or create intron and exon splice enhancer sites that affect alternative splicing activity as well as appropriate mRNA and protein production. Weka analysis with multiple classification paradigms on the 65 variances and subsets of these showed no specific model related to the 1-carbon folate pathway genes.

Stroke, a leading cause of morbidity, mortality, and increased health costs, has potential polygenic inheritance; the number of genes identified for stroke risk and pathophysiology is small and biological function of these identified genes does not explain pathophysiology. Technical approaches with GWAS have had limitations. The 1-carbon folate dependent pathway, including MTHFR, have polymorphisms and associated biomarkers that have been associated with stroke. This pathway may be useful for defining prevention and treatment targets for stroke; a combined platform of genes from this 1-carbon pathway based on biological knowledge, an early onset stroke dataset with GWAS and of preliminary candidate gene analysis by Plink with statistically significant SNPs, in combination with Weka data mining analysis and bioinformatics functional analysis has been used to look for and evaluate relevant stroke risk genes.

Stroke affects approximately 795,000 Americans each year, and approximately 6.4 million stroke survivors are now living in the United States. Although progress has been made in reducing stroke mortality, it is the fourth leading cause of death in the United States. However, stroke is the leading cause of disability in the United States and the rest of the world: 20% of survivors still require institutional care after 3 months and 15% to 30% experience permanent disability. Stroke is a life-changing event that also affects the patient's family members and caregivers.

Additionally in Germany, the projections for the period 2006 to 2025 showed 1.5 million and 1.9 million new cases of ischemic stroke in men and women, respectively, at a present value of 51.5 and 57.1 billion EUR, respectively. In Europe, stroke occurs in 7.2 individuals per 1000 per year (In Germany, 350/100000 people have a stroke yearly) with a short term mortality rate of 12%. This rises with age and with certain races and countries, being disproportionately higher in China, Africa, and South America, where stroke mortality may be at 27%. The risk for stroke may be significantly higher in those that have not had strokes, particularly in patients with hypertension, diabetes, obesity, and smoking use arguing for active prevention. For stroke, annual cost estimates for France and UK for stroke care are 2.5 billion Euros and 8.9 Billion pounds, which may be similar in Germany. Brain blood vessel imaging by magnetic resonance and computed tomographic imaging is expensive and not always reimbursable or accessible. Imaging is proactively needed to prevent and treat stroke.

Many important factors have contributed to current understanding of stroke. The definition of transient ischemic attack (TIA) has been revised and now excludes the patient whose acute neuroimaging findings reveal ischemia even if clinical symptoms have resolved. This change has shifted some formerly classified TIA patients into the category of ischemic stroke. An ischemic stroke is the result of neuronal death due to lack of oxygen, a deficit that produces focal brain injury. This event is accompanied by tissue changes consistent with an infarction that can be identified with neuroimaging of the brain. Strokes are usually accompanied by symptoms, but they also may occur without producing clinical findings and be considered clinically silent. Additionally, current transcranial imaging devices are severely limited by the aberrations caused by the skull.

Both acute and chronic conditions may result in cerebral ischemia or stroke. Acute events that can lead to stroke include cardiac arrest, drowning, strangulation, asphyxiation, choking, carbon monoxide poisoning, and closed head injury. More commonly, the etiology of stroke is related to chronic medical conditions including large artery atherosclerosis, atrial fibrillation, left ventricular dysfunction, mechanical cardiac valves, diabetes, hypertension and hyperlipidemia. Regardless of cause, prompt recognition of symptoms and urgent medical attention are necessary for thrombolytic therapy to be considered and provided.

Stroke or transient ischemic attacks (TIA) involve brain tissue damage that is permanent (stroke) or transient (TIA) from the obliteration of blood flow with reduced oxygen delivery through specific extracranial vessels, i.e. carotid arteries, cervical vertebral arteries, or intracranial vessels, i.e. middle cerebral arteries, posterior cerebral arteries due to atherosclerotic vessel change, emboli, or a combination of both. Emboli may be gaseous or particulate. The latter may involve calcium, fat, and blood elements including platelet, red blood cells, or organized clot, i.e. thrombin with platelets or thrombin alone. The size of these embolic components is approximately 50 microns for particulate or solid emboli and 1-10 microns for gaseous emboli. Particulate emboli may have a more important role in stroke or TIA causation, as compared to gas emboli; this underlies a need for detection and differentiation of particulate versus gas emboli.

Cerebral emboli may be associated with cardiac, aorta, neck and intracranial vessel disease, as well as coagulation disorders and neck and during diagnostic and surgical procedures on the heart and the carotid arteries. Cerebral embolism can be a dynamic process episodic, persistent, symptomatic, asymptomatic and may, but, not in all cases, predispose to stroke or TIA, influenced to some degree by composition and size; the latter embolic stroke, which is influenced by the vessel and its diameter to which the embolus goes.

Stroke is caused by reduced blood flow with reduced oxygen and nutrients which leads to irreversible damage to discrete or broad areas of brain with neurological deficits of varying degree and type. Stroke can be broadly classified as ischemic or hemorrhagic. Stroke is a leading cause of death, disability, and increased health care costs (428 Bentley, P. 2010). In the United States, it is estimated that 790,000 stroke cases occur yearly, including 200,000 cases of recurrent stroke. In the United States, the stroke mortality rate is 12% and the yearly stroke cost is 58 billion dollars (3993 Mohr, J. 2011). Worldwide, the stroke mortality rate ranges from 12% to 27% (3993 Mohr, J. 2011). Stroke incidence increases with age (3993 Mohr, J. 2011). Primary prevention by reduction or prevention of highly associated risk factors, such as diabetes, smoking, high blood pressure, obesity, and atrial fibrillation, may reduce stroke incidence and mortality. However, the identification of genes associated with stroke and stroke risk has the potential for diagnostic, preventive and therapeutic approaches to reduce stroke incidence and potentially ameliorate stroke morbidity and reduce stroke mortality.

Genetic factors that may be associated with stroke risk are difficult to ascertain because stroke occurrence is related to polygenic causes and environmental, epistatic, and lifestyle causes. The highly associated stroke risk factors of hypertension, smoking, and diabetes do not explain the broad variability in risk associated with the occurrence of these factors and stroke (428 Bentley, P. 2010). This underscores the complexity of identifying specific pathophysiologies in stroke. Genetic risk factors and their molecular products may play a role in this variability alone or in combination or in interaction with the common risk factors; genetic risk factors are important for “risk prediction and potential modification to reduce future events” (3926 Cronin, S. 2005; 2005). The common risk factors for stroke, i.e. atrial fibrillation, hypertension may have complex polygenic causes and, but also may be linked to certain genes (3844 Ellinor, P. T. 2012). However, “only a small proportion of ischemic stroke may be monogenic”, i.e. including but not limited to CADASIL with small vessel disease, familial hyperlipidemia, prothrombotic disorders (protein c deficiency, protein deficiency, factor V leiden) and mitochondrial disorders (MELAS) (1377 Markus H S 2010). Stroke is a complex polygenic disorder with epistatic/environmental and lifestyle influences (3853 Cole, J. W. 2011). Despite the lack of a single identified gene in most cases, stroke is heritable (3996 Casas, J. P. 2004) and is increased in monozygotic versus dizygotic twins (rev in) (3853 Cole, J. W. 2011). Further, stroke subtypes may have multiple or unique risk loci (3853 Cole, J. W. 2011). These include ischemic and hemorrhagic; the ischemic strokes can be broken down into 5 subtypes by TOAST criteria (Trial of Org 10172 in Acute Stroke Treatment) (3924 Marnane, M. 2010) using clinical and diagnostic criteria. These include large-artery atherosclerosis (Large vessel disease), cardioembolism, small-vessel occlusion, stroke of other determined etiology (rare, genetic causes), and stroke of undetermined etiology (3836 Hacke, W. 2012). These subtypes may have further intragroup heterogeneity. Stroke subtypes have distinct symptoms, distinct risk profiles, and subtype-specific risks or recurrent events or events in first degree relatives (3998 Grau, A. J. 2001) (3999 Kirshner, H. S. 2009). The complexity of the stroke phenotype and classification may increase the difficulty in studying the genetic factors involved in the etiology of stroke. Early onset stroke versus late onset stroke may have a stronger genetic contribution (3922 Brass, L. M. 1992) and more familial aggregation (3853 Cole, J. W. 2011); siblings of early onset stroke have a higher risk of stroke occurrence (3994 MacClellan, L. R. 2006). Parental risk of stroke by age 65 years increased the risk in offspring by 3 fold (3995 Seshadri, S. 2010). Age at onset has been associated in ischemic stroke siblings (409 Meschia, J. F. 2005). An early onset stroke dataset (3921 Cheng, Y. C. 2011) will be used in the current study.

### SUMMARY OF THE DISCLOSURE

Briefly stated, the present disclosure comprises method and device using a whole genome association analysis toolset for a total stroke case, versus a control group, which demonstrated significant associations with p less than 1.00E.03 for 5 genes, including polymorphisms in MTHFR [rs4846052 (intronic), rs7533315 (intronic), rs4846051 (intronic), rs6541003 (exonic)], MTRR (rs1802059), TYMS (rs2847149, rs2244500, rs1001761), BHMT (rs6893970), and SCL19A3 (rs13007334). 65 polymorphisms within the one-carbon folate genes were identified with p values less than 1.00E.01. MTHFR rs4846052 and rs7533315 were significantly associated with sex. The specific nucleotide polymorphism in MTHFR rs4846052 and rs7533315 intronic SNPs lead to removal of consensus Fox 1 and 2, intron splice enhancer sites (ISE); the MTHFR exonic SNP rs6864051, which is contiguous to rs1801131, a pathological SNP (A1298C) does not produce a projected pathological SNP but the nucleotide change removes a consensus exon splice enhancer site (ESS). These gene polymorphisms in MTHFR may remove or create intron and exon splice enhancer sites that affect alternative splicing activity as well as appropriate mRNA and protein production. Weka analysis with multiple classification paradigms on the 65 variances and subsets of these showed no specific model related to the 1-carbon folate pathway genes.

The present disclosure provides a method for administering a treatment to a human subject that reduces risk for early onset ischemic stroke, or a treatment to the human subject that assesses risk for early onset stroke, comprising: detecting the status (presence or absence) of one or more single nucleotide polymorphisms (SNPs) in genes selected from: SLC19A3 gene, methylenetetrahydrofolate reductase (MTHFR), thymidylate synthase (TYMS), methionine synthase reductase (MTRR), betaine homocysteine S-methyltransferase (BHMT), and folate receptor 2 (FOLR2), wherein the one or more SNPs is selected from: rs7533315; rs4846052; rs6541003; rs4846051; rs1802059; rs6893970; SNP2-228286391; rs13007334; rs1001761; rs2847149; rs2244500; rs229844 (“SNP list”), wherein the SNPs were identified in a database using Early Age Onset Stroke Association Algorithm, followed by the step of implementing a treatment that uses the detected status (presence of SNP or absence of SNP), wherein if the subject comprises the SNP, administering one or both of: (i) treatment that reduces risk for early onset stroke, or (ii) treatment that is diagnostic for assessing risk for early onset stroke.

Also provided, is the above method, further comprising the steps of withdrawing at least one cell from the human subject, and processing the at least one cell to provide a source of genomic DNA suitable for identifying single nucleotide polymorphisms (SNPs).

Further encompassed, is the above method, wherein the treatment that diagnoses risk for early onset stroke comprises one or more of stimulating the tissues of the subject's body to vibrate using ultrasound vibration, stimulating the excitation of hydrogen atoms in the subject's body using Magnetic Resonance Imaging (MRI), and causing ionization of organic molecules in the subject's body using computed tomography (CT).

Moreover, what is also encompassed is the above method, wherein the treatment that reduces risk for early onset stroke comprises administering one or more of folic acid, vitamin B12, vitamin B6, thiamin, aspirin, platelet antagonist, blood clotting antagonist, HDL cholesterol reducing agent, anti-hypertriglyceridemia agent, and anti-hypertensive agent.

Further contemplated is the above method, wherein the one or more SNPs does not comprise any SNP that is not the SNP list.

Also embraced, is the above method, wherein the one or more SNPs comprises at least two SNPs selected from the SNP list, and that further comprises at least one SNP that is not in the SNP list.

Additionally provided is the above method, wherein the one or more SNPs comprises at least two SNPs selected from the SNP list, wherein at least one of the at least two SNPs selected from the SNP list is a SNP that is homozygous in the patient's genome.

Also embraced is the above method, wherein the one or more SNPs comprises at least two SNPs selected from the SNP list, wherein at least two of the at least two SNPs selected from the SNP list is a SNP that is homozygous in the patient's genome.

In another embodiment, what is provided is the above method wherein the one or more SNPs comprises at least three SNPs selected from the SNP list, wherein at least three of the at least two SNPs selected from the SNP list is a SNP that is homozygous in the patient's genome.

Further provided is the above method, wherein the one or more SNPs comprises at least two SNPs selected from the SNP list, wherein at least one of the at least two SNPs selected from the SNP list is associated with abnormal splicing.

Also provided is the above method, wherein the one or more SNPs comprises at least two SNPs selected from the SNP list, wherein at least two of the at least two SNPs selected from the SNP list is associated with abnormal splicing.

In yet another embodiment, what is provided is the above method, wherein the one or more SNPs comprises at least three SNPs selected from the SNP list, wherein at least three of the at least two SNPs selected from the SNP list is associated with abnormal splicing.

In another aspect, what is provided is the above method wherein the single nucleotide polymorphism is a mutation is in one or more of a coding region of the gene, a promoter of the gene, a splicing region of the gene, an enhancer of the gene, an intron, a region of the chromosome corresponding to an upstream untranslated region (UTR), a region of the chromosome corresponding to a downstream untranslated region (UTR), or a region characterized by simultaneous residence in two different genes where one gene resides in Watson strand, and the other gene resides in Crick strand.

Also provided is the above method, further comprising assessing status for at least one vitamin concurrently with said detecting the status of one or more single nucleotide polymorphisms (SNPs) in genes.

Moreover, what is also provided is the above method, further comprising assessing status for at least one vitamin concurrently with said detecting the status of one or more single nucleotide polymorphisms (SNPs) in genes, wherein the status for at least one vitamin comprises assessing status for one or more of folate, vitamin B6, vitamin B12, and thiamin.

In another aspect, what is provided is the above method, wherein the method is not used for a subject that comprises hemorrhagic stroke, or not used for a subject that comprises late onset stroke.

Further encompassed is the above method, wherein the one or more SNPs comprises at least two SNPs selected from the SNP list, wherein at least one of the at least two SNPs selected from the SNP list is a SNP that is heterozygous in the patient's genome.

Also contemplated is the above method, wherein the one or more SNPs comprises at least two SNPs selected from the SNP list, wherein at least two of the at least two SNPs selected from the SNP list is a SNP that is heterozygous in the patient's genome.

In another methods embodiment, what is provided is a method for administering a treatment to a human subject that reduces risk for early onset stroke, or a treatment to the human subject that assesses risk for early onset stroke, comprising: withdrawing at least one cell from the human subject, and processing the at least one cell to provide a source of genomic DNA suitable for identifying single nucleotide polymorphisms (SNPs), detecting the status (presence or absence) of one or more single nucleotide polymorphisms (SNPs) in genes selected from: SLC19A3 gene, methylenetetrahydrofolate reductase (MTHFR), thymidylate synthase (TYMS), methionine synthase reductase (MTRR), betaine homocysteine S-methyltransferase (BHMT), and folate receptor 2 (FOLR2), wherein the one or more SNPs is selected from: rs7533315; rs4846052; rs6541003; rs4846051; rs1802059; rs6893970; SNP2-228286391; rs13007334; rs1001761; rs2847149; rs2244500; rs229844, wherein the SNPs were identified in a database using Early Age Onset Stroke Association Algorithm.

In another aspect, what is provided is the above method, followed by the step of implementing a treatment that uses the detected status (presence of SNP or absence of SNP), wherein if the subject comprises the SNP, administering one or both of: (i) treatment that reduces risk for early onset stroke, or (ii) treatment that is diagnostic for assessing risk for early onset stroke.

Moreover, what is also provided is the above method, wherein the method is not used with a subject that comprises hemorrhagic stroke.

In a systems embodiment, what is provided is a system comprising a computer that is configured to apply the Early Stroke Algorithm to identify Single Nucleotide Polymorphisms (SNPs) that are significantly associated with risk for early onset stroke, wherein the significance of the association has a P value of less than 0.05. Also provided is the above system, wherein the significance of the association has a P value that is less than 0.005, or is less than 0.002, or is less than 0.001, or is less than 0.0005, or is less than 0.0002, or is less than 0.0001, and so on.

### LIST OF TABLES

Table 1. Abbreviations.

Table 2. GeneBank accession numbers.

Table 3. Weka analysis.

Table 4. Weka analysis.

Table 5. Weka analysis.

Table 6. Decision tree.

Table 7. Thirteen (13) SNPs of the present disclosure, where one of the SNPs (rs1801133*) is an example of a SNP with relatively low P value.

Table 8. Further information on SNP of the present disclosure.

Table 9. Homogeneous and heterogeneous SNP alleles.

Table 10A-10J. Combinations of the twelve (12) SNPs of greatest statistical significance for the present system and methods. The combinations set forth in the tables also include one SNP that is of lesser statistical significance (rs1801133).

Table 11. Population characteristics of the GENEVA database.

Tables 12A, B, C, D, E, F. Details of Plink analysis

### ABBREVIATIONS

TABLE 1
Abbreviations

| | |
|---|---|
| BHMT | Betaine homocysteine S-methyltransferase |
| CADASIL | Cerebral Autosomal-Dominant Arteriopathy |
| | with Subcortical Infarcts and |
| | Leukoencephalopathy is a hereditary stroke |
| | disorder |
| 1-Carbon metabolism | The net collection of anabolic and catabolic |
| | pathways involving the transfer of 1-carbon |
| | units, e.g., in the biosynthesis of thymidylate, |
| | and in the regeneration of methionine. |
| FOLR2 | Folate receptor 2; folate receptor beta |
| GEOS | Genetics of Early Onset Stroke |
| GWAS | Genome-Wide Association Studies |
| MELAS | Mitochondrial Encephalomyopathy, Lactic |
| | Acidosis, and Stroke-like episodes |
| MTHFR | Methylenetetrahydrofolate reductase |
| MTR | Methionine synthase; 5- |
| | methyltetrahydrofolate-homocysteine |
| | methyltransferase |
| MTRR | Methionine synthase reductase |
| REC | Reduced folate carrier |
| SLC19A3 | Thiamine transporter 2 (ThTr-2); solute carrier |
| | family 19 member 3. The SLC19 family of |
| | transporters mediates transport of folates |
| | (Eudy et al (2000) Mol. Genet. Metab. |
| | 71: 581-590; Zhao and Goldman (2013) Mol. |
| | Aspects Med. 34: 373-385). |
| SHMT | Serine hydroxymethyltransferase |
| SNP | Single nucleotide polymorphisms |
| SPYM | Stroke Prevention in Young Men |
| SPYW-1 | Stroke Prevention in Young Women |
| THF | tetrahydrofolate |
| TOAST criteria | Trial of ORG 10172 in Acute Stroke Treatment |
| | (TOAST) criteria. See, Marnane et al (2010) |
| | Stroke. 41: 1579-1586 |
| TIA | Transient ischemic attack |
| TYMS | Thymidylate synthase |

### GenBank Sequences of Human Genes

TABLE 2
GenBank sequences of human genes, or publications
disclosing gene sequence

| | |
|---|---|
| BHMT | NM_001713.2 |
| FOLR2 | NM_000803.4; NM_001113534.1; |
| | NM_001113535.1; NM_001113536.1 |
| MTHFR | NM_005957.4 |
| MTR | U75743.1; U73338.1 |
| MTRR | Leclerc et al (1998) Proc. Natl. Acad. Sci. |
| | 95: 3059-3064 |
| REC | AH006305.1 |
| SLC19A3 | AF283317.1; NM_025243.3 |
| SHMT | L23928.1 |
| TYMS | AB077207.1; D00596.1 |

### BRIEF DESCRIPTIONS OF THE FIGURES

FIG. 1 discloses pathways of 1-carbon metabolism. FIG. 1A shows folate-mediate biosynthesis of purines, biosynthesis of thymidylate, and remethylation of methionine. Most of these methyl groups are donated by serine. FIG. 1B show steps where 1-carbon units are added to THF, thereby regenerating 5-methyl-THF, and where S-adenosyl-homocysteine is receives 1-carbon units, thereby regenerating S-adenosyl-methionine (SAM). SAM donates methyl groups in a large number of methylation reactions in the body.

FIG. 2 illustrates the steps of reading TPED file, extracting information, processing the data, transposing the records, categorizing the data, and including case, control variables in the data.

FIG. 3 shows the platform for Plink and Weka analysis.

FIG. 4 shows P values for eleven (11) SNPs.

FIG. 5 shows map of MTHFR, revealing locations of exons, introns, and SNPs.

### DETAILED DESCRIPTION

The present disclosure encompasses all possible combinations of the above embodiments, and encompasses all possible disclosures of each independent claim with its dependent claims. For example, what is encompassed is an invention that is the combination of: Claim 1+Claim 2; or the combination of: Claim 1+Claim 2+Claim 3; or the combination of Claim 1+Claim 3+Claim 4; or the combination of Claim 1+Claim 2+Claim 3+Claim 4; and the like.

As used herein, including the appended claims, the singular forms of words such as “a,” “an,” and “the” include their corresponding plural references unless the context clearly dictates otherwise. All references cited herein are incorporated by reference to the same extent as if each individual publication, patent, and published patent application, as well as figures and drawings in said publications and patent documents, was specifically and individually indicated to be incorporated by reference.

The terms “adapted to,” “configured for,” and “capable of,” mean the same thing. Where more than one of these terms is used in a claim set, it is the case that each and every one of these terms, as they might occur, means, “capable of.”

### The Novel “Early Age Onset Stroke Association Algorithm”

Using a quality dataset (GENEVA) on early onset stroke (15-49 years) and biological knowledge suggesting the one-carbon folate dependent pathway, including MTHFR as a potential contributor to stroke risk, extraction of the data from the dataset on 22 genes within the one-carbon folate pathway using a specially designed Perl program. The data from the Geneva dataset on these 22 genes was then loaded into Plink. Association analysis with multiple measures and logistic regression using Plink was done on 22 genes and their variants comparing cases with stroke and controls. one-carbon Pathway gene variants that reached a threshold of significance greater than 0.02. Thirteen specific SNPs from the 1-carbon folate pathway were identified that were significantly different between controls and early onset stroke cases. These specific SNPs related to specific genes within the 1-carbon folate pathway were identified and further evaluated to predict SNP variance sequence function by analysis of the biological function of their respective genes and with PolyPhen-2 and with analysis of gene structure and function with GenBank, dbSNP, BLAST Aceview, Exon Scan and Haploview. Using Plink Association and Model (Trend, Allelic, Dominant, Recessive) analysis, the total stroke case versus the control group demonstrated significant associations with p less than 0.001 for 5 genes with 13 SNPs, including polymorphisms in MTHFR [rs4846052 (intronic), rs7533315 (intronic), rs4846051 (intronic), rs6541003 (exonic)], MTRR (rs1802059), TYMS (rs2847149, rs2244500, rs1001761), BHMT (rs6893970), SLC19A3 (rs13007334) and FoIR2 (rs229844). 65 polymorphisms within the one-carbon folate genes were identified with p values less than 0.01. Analysis of the nucleotide sequence within 30 basepairs 5′ and 3′ including the wild type and abnormal SNP, for each of the 13 SNPs was then done to look for alteration in consensus splice sites and intron and exon splicing enhancers and silencers, and alternative splicing motifs. The specific nucleotide polymorphism in MTHFR rs4846052 and rs7533315 intronic SNPs lead to removal of consensus Fox 1 and 2, intron splice enhancer sites (ISE); the MTHFR exonic SNP rs6864051, which is contiguous to rs1801131, a pathological SNP (A1298C) does not produce a projected pathological SNP but the nucleotide change removes a consensus exon splice enhancer site (ESS). These gene polymorphisms in MTHFR may remove or create intron and exon splice enhancer sites that affect alternative splicing activity as well as appropriate mRNA and protein production.

TPED data from all SNPs that had a Plink association of p less than 0.01 or less than 0.001 were then processed with a specially designed C++ program to get base-pair information, transposed with excel, and individual significance groups were analysed with controls versus cases were then evaluated for multi gene interaction within the 1-carbon folate pathway using data mining techniques such as Random forest and naïve Bayes classifiers with Weka. Weka analysis with multiple classification paradigms on the 65 variances and subsets of these showed no specific model related to the one-carbon folate pathway genes. Thus, an integrated platform for the public dataset with a PERL extraction code for the specific genes, importation and analysis in PLINK, selection and conversion of significant gene associations with C++ and excel into a CSV file, and then integration and loading to Weka has been utilized and developed.

This novel integrated platform using bioinformatics with a combinatorial approach of biological pathway knowledge with stroke relevance combined with an early onset stroke dataset, the public GENEVA dataset, Plink association statistics, logistic regression, and multilocus classifiers. This integrated platform can be used to identify novel common gene variants in a polygenic complex disorder, such as stroke and a preliminary screen to identify genes for further evaluation of functional and biological significance and relevance. Variants in multiple genes in the one-carbon folate derived pathway may act alone or in combination to increase stroke risk and may predispose to stroke in a general manner, regardless of stroke subtype; these SNPs may be involved in a novel and new but potentially common mechanism for enhancement or silencing of alternative splicing within exons and introns in methylation economy enzymes/genes, at least in this pathway, that may contribute to stroke pathophysiology and risk. These novel, previously unreported SNPs for MTHFR, BHMT, MTRR, and TYMS, that are primary regulators of methylation and homocysteine metabolism were identified with a potential useful platform that employed an integrated approach with focused data extraction from a large public dataset, its use in Plink, extraction and processing for Weka analysis for combined GWAS and data mining followed by molecular functional analysis using bioinformatics. In combination with clinical and neurological history and examination, identified medical disorders, relevant family history, relevant brain and neck imaging studies and other routine and warranted diagnostic tests, the specific genes and their SNPs identified here in the 1-carbon folate pathway and putative abnormalities in splicing function may provide a basis for finding patients at increased risk for stroke or recurrent stroke and assisting in the management of these patients with vitamins, co-factors, and other therapies to reduce stroke occurrence and recurrence.

### Commentary on the Novel Algorithm

The novel algorithm of the present disclosure is a tool for pre-selecting an important pathway, or genes that mediate steps in that metabolic pathway, that are relevant to early stroke. The algorithm provides for screening a large undifferentiated dataset for important SNPs that predispose to early stroke. As such, the algorithm of the present disclosure uses novel software tools that the present inventors have designed for use with existing instruments, brought together in a logical bioinformatic set of steps. The present tools allow identification of potential SNPs of genes that have functional importance, and whose change may alter function and predispose to early stroke, by altering 1-carbon metabolism. For example, the SNP can alter the regulation of protein expression, alter the catalytic rate of an enzyme, or alter the regulatory properties of that enzyme.

A method and device using a whole genome association analysis toolset for a total stroke case, versus a control group, which demonstrated significant associations with p less than 1.00E.03 for 5 genes, including polymorphisms in MTHFR [rs4846052 (intronic), rs7533315 (intronic), rs4846051 (intronic), rs6541003 (exonic)], MTRR (rs1802059), TYMS (rs2847149, rs2244500, rs1001761), BHMT (rs6893970), and SCL19A3 (rs13007334). 65 polymorphisms within the one-carbon folate genes were identified with p values less than 1.00E.01. MTHFR rs4846052 and rs7533315 were significantly associated with sex. The specific nucleotide polymorphism in MTHFR rs4846052 and rs7533315 intronic SNPs lead to removal of consensus Fox 1 and 2, intron splice enhancer sites (ISE); the MTHFR exonic SNP rs6864051, which is contiguous to rs1801131, a pathological SNP (A12980) does not produce a projected pathological SNP but the nucleotide change removes a consensus exon splice enhancer site (ESS). These gene polymorphisms in MTHFR may remove or create intron and exon splice enhancer sites that affect alternative splicing activity as well as appropriate mRNA and protein production. Weka analysis with multiple classification paradigms on the 65 variances and subsets of these showed no specific model related to the 1-carbon folate pathway genes.

Table 3 discloses Weka Analysis [9] by Naïve Bayes of SNPs (223) with p values less than 0.1 (Table 3), SNPs (7) with p values less than 0.01 (Table 4), and SNPs (Chromosome 1, enriched for MTHFR) with values less than 0.001(Table 5), less than 0.01 (Chromosome 1) (not shown) and less than 0.1 (Chromosome 1) (not shown). The correctly classified instances were not greater than random chance for Naïve Bayes and other Weka classifiers for all SNP significance groups specific gene group by significance level threshold.

TABLE 3
Weka analysis

| | |
|---|---|
| Naïve Bayes of SNPs with p values less than 0.1 | |
| Stratified cross-validation | |
| Correctly classified instances 975 | 53.7486% |
| Incorrectly classified instances 839 | 46.2514% |
| Kappa statistic 0.0727 | |
| Mean absolute error 0.4648 | |
| Root mean squared error 0.5747 | |
| Relative absolute error 93.0192% | |
| Root relative squared error 114.9741% | |
| Total number of instances 1814 | |

Detailed accuracy by class

| | TP | FP | | | | ROC | |
|---|---|---|---|---|---|---|---|
| | rate | rate | Precision | Recall | F-measure | area | Class |
| | 0.486 | 0.413 | 0.528 | 0.486 | 0.506 | 0.563 | 0 |
| | 0.587 | 0.514 | 0.545 | 0.587 | 0.565 | 0.563 | 1 |
| weighted | 0.537 | 0.465 | 0.537 | 0.537 | 0.536 | 0.563 | |
| average | | | | | | | |

Confusion matrix

| a | b | <-- classified as |
|---|---|---|
| 430 | 455 | a = 0 |
| 384 | 545 | b = 1 |

TABLE 4
Weka analysis

| | |
|---|---|
| Stratified cross-validation | |
| Correctly classified instances 972 | 53.5832% |
| Incorrectly classified instances 842 | 46.4168% |
| Kappa statistic 0.069 | |
| Mean absolute error 0.483 | |
| Root mean squared error 0.5102 | |
| Relative absolute error 96.6477% | |
| Root relative squared error 102.0788% | |
| Total number of instances 1814 | |

Detailed accuracy by class

| | TP | FP | | | | ROC | |
|---|---|---|---|---|---|---|---|
| | rate | rate | Precision | Recall | F-measure | area | Class |
| | 0.476 | 0.407 | 0.527 | 0.476 | 0.5 | 0.557 | 0 |
| | 0.593 | 0.524 | 0.543 | 0.593 | 0.567 | 0.557 | 1 |
| weighted | 0.536 | 0.467 | 0.535 | 0.536 | 0.534 | 0.557 | |
| average | | | | | | | |

Confusion matrix

| a | b | <-- classified as |
|---|---|---|
| 421 | 464 | a = 0 |
| 378 | 551 | b = 1 |

TABLE 5
Weka analysis

| | |
|---|---|
| Stratified cross-validation | |
| Correctly classified instances 975 | 53.7486% |
| Incorrectly classified instances 839 | 46.2514% |
| Kappa statistic 0.0682 | |
| Mean absolute error 0.4933 | |
| Root mean squared error 0.4986 | |
| Relative absolute error 98.7106% | |
| Root relative squared error 99.7451% | |
| Total number of instances 1814 | |

Detailed accuracy by class

| | TP | FP | | | | ROC | |
|---|---|---|---|---|---|---|---|
| | rate | rate | Precision | Recall | F-measure | area | Class |
| | 0.385 | 0.318 | 0.536 | 0.385 | 0.448 | 0.543 | 0 |
| | 0.682 | 0.615 | 0.538 | 0.682 | 0.602 | 0.543 | 1 |
| weighted | 0.537 | 0.47 | 0.537 | 0.537 | 0.527 | 0.543 | |
| average | | | | | | | |

Confusion matrix

| a | b | <-- classified as |
|---|---|---|
| 341 | 544 | a = 0 |
| 295 | 634 | b = 1 |

### Ultrasound

The present disclosure provides for ultrasonography of vessels located in (or pass through) the neck, in the brain, or of vessels that are located in both neck and brain. These vessels include carotid arteries, external carotid artery, vertebral arteries, basilar artery, bilateral innominate artery, subclavian artery, subclavian vein. Equipment for ultrasound includes, e.g., Acuson Sequoia system from Siemens AG, Healthcare Sector (Erlangen, Germany) or HHU system (Sonosite MicroMaxx, Bothell, Wash.). Guidance for conducting sonography is available, e.g., from Kim et al (2010) J. Ultrasound Med. 29:1161-1165; Rubin et al (2010) J. Ultrasound Med. 29:1385-1390, Johnson et al (2011) J. Am. Son. Echocardiogr. 24:738-747.

Methods that can be Integrated with SNP Test of the Present Disclosure

The present disclosure provides a SNP test based on genes that mediate 1-carbon metabolism, where this test is a step in an integrated method that further comprises contacting a human subject with one or more of a device, ultrasound vibrations, irradiation, administered contrast media, and so on, using one of the following diagnostic machines. The diagnostic machines include Duplex ultrasound (DUS), which images carotid stenosis, intra-arterial angiography (IAA), non-contrast magnetic resonance angiography (MRA), and contrast enhanced computed tomographic angiography (CTA) (Khan et al (2007) J. Neurol. Neurosurg. Psychiatry. 78:1218-1225). Diagnostic machines include non-contrast head computed tomography (CT), which is used for diagnosing subarachnoid hemorrhage (Connolly, Rabinstein, Carhuapoma et al (2012) Stroke. 43 (27 pages). The systems and methods of the present disclosure encompass contacting a human subject with vibrotactile noise, for example, for enhancing touch sensation in stroke survivors (Enders et al (2013) J. NeuroEngineering Rehabilitation. 10:105 (8 pages)).

The system and methods of the present disclosure can be conducted with a human subject who lacks a medical history that establishes risk for early stroke. In addition, the system and methods of the present disclosure can be conducted with a human subject who has a history of, e.g., atrial fibrillation, previous stroke, coronary artery disease, diabetes mellitus, hypertension, dyslipidemia, smoker, obesity, diet that is high in saturated fat, and the like. Where a subject presents with a medical history that includes one or more of the above, and also where the subject's medical history does not include one of the above, the system and methods of the present disclosure provide an additional and independent measure of risk.

Human Subject of the Present Disclosure Possessing Homozygous SNP or Heterozygous SNP

In some embodiments, the system and method of the present disclosure use homozygous SNPs, in a decision tree, resulting in a go decision. In other embodiments, the system and methods will use heterozygous SNPs, in a decision tree, resulting in a go decision. Where a mutation is homogeneous rather than hetergeneous, such as a mutation that is a SNP in a gene that mediates 1-carbon metabolism and that results in a splicing defect, the detection of the homogeneous SNP will mean that the human subject is at greater risk for early onset stroke. In contrast, the detection of a SNP that is merely heterogeneous, will indicate risk for early onset stroke, but a risk that is somewhat lesser than where the SNP mutation is homogeneous. For application of the algorithm and decision tree of the present disclosure, a go decision can be based only where there is least one homogeneous SNP (when the patient's genome is subjected to an interrogation by one or more of the novel SNPs identified by the present disclosure). Alternatively, the go decision can be made where all of the SNPs in the patient's genome are homogeneous (this statement applies to the SNPs that are used in the present system and method). Also, alternatively, the go decision can be made where the patient's genome has at least two homogeneous SNPs, at least three, at least four, at least five, at least six, at least seven at least eight, at least nine, at least ten, at least 11, at least 12, at least 13 homogenous SNPs. In exclusionary embodiments, the system and method can exclude any method that takes into account SNPs that are heterogenous (rather than being homogeneous).

### Exclusionary Embodiments Relating to Age

The present disclosure can, in some embodiments, exclude any SNP that was identified as indicating risk for stroke, where the research study indicating this risk, use a population of subjects that was substantially 55 years of age or older. Also, what can be excluded is any SNP, where the population of subjects consisted of at least 10% older than 55 years of age, at least 20% older, at least 30% older, at least 40% older, at least 50% older, at least 60% older, at least 70% older, at least 80% older, at least 90% older than 55 years of age. Also, what can be excluded is any SNP, where the population of subjects consisted of at least 10% older than 60 years of age, at least 20% older, at least 30% older, at least 40% older, at least 50% older, at least 60% older, at least 70% older, at least 80% older, at least 90% older than 60 years of age. Also, what can be excluded is any SNP, where the population of subjects consisted of at least 10% older than 65 years of age, at least 20% older, at least 30% older, at least 40% older, at least 50% older, at least 60% older, at least 70% older, at least 80% older, at least 90% older than 65 years of age.

### Decision Tree

The following table provides a non-limiting decision tree for the system and method of the present disclosure.

TABLE 6
Decision tree. Early Stroke Risk Decision Tree for identification,
continuous patient follow-up, risk factor identification, and potential
treatment to prevent first or subsequent stroke

Age less than or equal to 55 years
Yes No. (If age is less than or equal to 55 years, then continue to follow this decision
tree)
Diabetes (Chemical or treated)
History of Hypertension
Blood pressure (BP) systolic greater than 140 or Diastolic blood pressure greater than
or equal to 90
Black race
Hispanic/Latino American
Positive family history of stroke
Patient history of stroke or transient ischemic attack
Abnormal neurological examination that can be related to specific neck or brain
circulation territory
Elevation of blood homocysteine
Family history or patient history of coagulation disorder, including but not limited to
protein C and S deficiency, Factor V leiden
Patient history of collagen vascular disease or anti-cardiolipin antibody syndrome
Abnormal carotid Doppler examination with stenosis or abnormal plaque
Abnormal Brain CT or MRI that suggests stroke
Abnormal MR or CT angiogram of neck or brain
Inherited genetic disorder that may predispose to stroke (Fabry's disease, CADASIL
High total cholesterol
Low HDL cholesterol
Atrial Fibrillation, Left atrial thrombus, prosthetic cardiac valve
Coronary Artery Disease or peripheral vascular diseae
Previous Myocardial Infarction
Congestive Heart Failure
Obesity
Abnormal BMI
Sickle Cell Disease
Migraine with Aura, Basilar Migraine, Hemiplegic Migraine
Metabolic Syndrome
Alcohol consumption (greater than or equal to 5 drinks per day)
Drug Abuse
Chronic smoking, particularly cigarette
Sleep disordered breathing, including Sleep Apnea
Postmenopausal hormone therapy
Elevated lipoprotein
Elevated CRP
Oral contraceptives
Elevated independent first stroke risk score that includes age, systolic blood pressure,
diabetes, current smoking, established cardiovascular disease, atrial fibrillation, and left
ventricular hypertrophy on electrocardiogram (Framingham Stroke Profile)
(If one or more of the above conditions is met by the human subject, then continue to
follow this decision tree.)
Yes No
Modify and treat above conditions to reduce stroke risk or stroke recurrence, including
but not limited to high blood pressure, cigarette smoking, diabetes, dyslipidemia, atrial
fibrillation, other cardiac conditions, asymptomatic carotid stenosis, sickle cell disease,
postmenopausal hormone therapy, oral contraceptives, reduced intake of sodium and
potassium, physical activity optimization, obesity, migraine, alcohol consumption, drug
abuse, sleep disordered breathing, elevated homocysteine, hypercoagulability,
elevated Lp(a), metabolic syndrome
Appropriate antiplatelet, anticoagulant, and statin therapy as specifically determined
If warranted, additional imaging of brain with MRI and/or CT scan of brain, MR or CT
angiography of head and neck, transcranial Doppler with and without emboli detection,
carotid Doppler and with therapy appropriate for these study results and patient clinical
and neurological history and examination
Yes No
Blood screening for hypercoagulable disorders, collagen vascular disorder, anti
cardiolipin antibody syndrome, as warranted
Abnormal
Yes No
Initiate therapy
Blood screening for 1-Carbon folate pathway metabolites and co-factors, including but
not limited to homocysteine, folate, B12, B6, thiamine, other vitamins, and other
metabolites
Blood screening for 1-carbon folate pathway population specific early stroke
polymorphisms, including but not limited to MTHFR, MTRR, BHMT, TYMS, FolR2, and
SCL19A3
Yes No
Abnormal screening of 1 carbon folate pathway metabolites, co-factor deficiency, or
abnormal gene polymorphism presence in homozygotic or heterozygote state
Initiate vitamin and other preventive therapies to ameliorate abnormalities in 1 carbon
folate pathway components
Yes No
Follow blood levels of 1 carbon folate pathway metabolites or co-factors
Abnormal
Yes No
Initiate corrective therapy. This corrective therapy is in addition to any corrective
therapy that has been previously initiated, or that is already been administered.

### Details of Decision Tree

The following provides for a decision tree that can be used in the present system and method. There is sequence based on the decision tree that needs to be gone thru before going forward and incurring the cost. The SNP test, in a non-limiting embodiment of the present disclosure, is done concurrently with the blood levels of folate and other key vitamins in the pathway would be run for levels. Pending the levels and the identification of SNp13 plus doing a MTHFR C677T, then folate, B12, thiamine would be given. However, the amount given would depend on the level found and the potential defect. The ultrasound of the neck, CT scan, transcranial Doppler, MRI, etc. should be done based on the neurological examination and history and the other risk factors and depending on the clinical situation and judgment of the clinician, in combination with the SNP13 screening.

Defining Set-Points in Datasets, Prior to Exploring the Dataset for SNPs that are Associated with Early Onset Stroke

The following defines set-points that can be used, where an dataset is explored for SNPs that are associated with early onset stroke, and that are predictive for early onset stroke. In evaluating the GENEVA dataset, or any other dataset, any given SNP was selected for use in the list where analysis of the data satisfied one of the following criteria. Shown below are a dozen different criteria:

(i) The SNP occurred in the genome of at least 5.0% of the early stroke victim group (population), and in less than 5.0% of the normal control group (population); (ii) The SNP occurred in the genome of at least 2.0% of the early stroke victim group, and in less than 2.0% of the normal control group; (iii) The SNP occurred in the genome of at least 1.0% of the early stroke victim group, and in less than 1.0% of the normal control group; (iv) The SNP occurred in the genome of at least 0.5% of the early stroke victim group, and in less than 0.5% of the normal control group; (v) The SNP occurred in the genome of at least 0.2% of the early stroke victim group, and in less than 0.2% of the normal control group; (vi) The SNP occurred in the genome of at least 0.1% of the early stroke victim group, and in less than 0.1% of the normal control group; (vii) The SNP occurred in the genome of at least 0.05% of the early stroke victim group, and in less than 0.05% of the normal control group; (viii) The SNP occurred in the genome of at least 0.01% of the early stroke victim group, and in less than 0.01% of the normal control group; (xi) The SNP occurred in the genome of at least 0.005% of the early stroke victim group, and in less than 0.005% of the normal control group; (x) The SNP occurred in the genome of at least 0.002% of the early stroke victim group, and in less than 0.002% of the normal control group; (xi) The SNP occurred in the genome of at least 0.001% of the early stroke victim group, and in less than 0.001% of the normal control group;

Where the SNP occurred in both chromosomes (homozygosity) of a particular early stroke victim, the datum point was considered to be from two different early stroke victim subjects. In other words, the weight given to that datum point was doubled. (Where the SNP in question was homozygous in a particular early stroke victim subject, this result further encouraged us to include the SNP in our SNP list.)

Also, where the SNP occurred in both chromosomes (homozygosity) of a particular normal control subject, the datum point was considered to be from two different normal control subjects. In other words, the weight given to that datum point was doubled. (Where the SNP in question was homozygous in a particular normal control subject, this result teaches against including that particular SNP in our list, or in other words, reduces the weight of that particular SNP as a predictive factor.)

Dietary Practices in Studies of Cardiovascular Disease

Populations of study subjects, for example, for studies of early stroke, can be classified according to criteria, such as dietary practices, gender, ethnicity, geographical location of residence, age, income group, and so on. Regarding dietary practices, for example, studies of populations in Baltimore, Md. have been characterized as having a disproportionately high levels of obesity and diet-related chronic diseases, stemming from consumption of high-fat foods, and prevalence of fast food and carry-out stores that market high-fat foods (Gittelsohn et al (2010) Health Promot. Pract. 11:723-732). Fruit and vegetable intake in Baltimore, Md. has been reported to be extremely low, and obesity prevalence has been determined to be about 60 percent (Lee et al (2010) Ecol. Food Nutr. 49:409-430). To provide another example, epidemiological studies in Italy have demonstrated that the foods typically available in Italy are correlated with the greatest reduction in risk for stroke, as compared with populations consuming other well-characterized diets (Agnoli et al (2011) J. Nutr. 141:1552-1558). In addition to the Italian Mediterranean diet, Agnoli et al studied subjects consuming the Greek Mediterranean diet, Healthy Eating Index diet, and Dietary Approaches to Stop Hypertension (DASH) diet. The study subjects of Agnoli et al consisted of about 47,000 Italian subjects. The Mediterranean diet has been reviewed (see, e.g., Trichopoulou (2004) Public Health Nutr. 7:943-947; Sofi et al (2010) Am. J. Clin. Nutr. 92:1189-1196). Regarding differences in early onset stroke in populations in Baltimore and Italy, Kittner et al (1993) Stoke. 24 (12 Suppl.) 113-115, reported that cerebral infarction rates are high in Baltimore, and relatively low in Florence, Italy. In young adults in Baltimore, cerebral infarction rates per 100,000 were 22.8 for black males, 10.3 for white males, 20.7 for black females, and 10.8 for white females. This study of young adults further reported intracerebral hemorrhage rates per 100,000 were 14.2 for black males, 4.6 for white males, 4.8 for black females, and 1.5 for white females.

Genes that Mediate 1-Carbon Metabolism

In a non-limiting embodiment, genes that are used to mediate 1-carbon metabolism include a homologous naturally-occurring gene, where the encoded polypeptide has at least 30% amino acid sequence identity to the encoded polypeptide of a gene that has a proven role in mediating 1-carbon metabolism, such as a folate transporter, a regulatory protein, or a folate-requiring enzyme. Polypeptide sequence identity analysis using LALIGN program, available on EXPASY, was performed on the following three polypeptides of the SLC19A family:

NM_{—}194255; 2873 bp; mRNA; linear PRI 02-SEP-2013; Homo sapiens solute carrier family 19 (folate transporter), member 1 (SLC19A1), transcript variant 1, mRNA.

NM_{—}006996; 3655 bp; mRNA; linear PRI 19-AUG-2013; Homo sapiens solute carrier family 19 (thiamine transporter),member 2 (SLC19A2), mRNA.

AF283317; 3532 bp; mRNA; linear PRI 02-JUL-2001; Homo sapiens orphan transporter SLC19A3 (SLC19A3) mRNA, complete cds.

LALIGN analysis between SLC19A1 and SLC19A2 revealed 39.7% sequence identity, 464 amino acid overlap (25-465; 30-489). LALIGN analysis between SLC19A1 and SLC19A3 revealed 42.5% sequence identity, 452 amino acid overlap (24-451; 11-458). LALIGN analysis between SLC19A2 and SLC19A3 revealed 53.2% sequence identity, 444 amino acid overlap (30-471:12-454) (ExPASy Bioinformatics Resource Portal, Swiss Institute of Bioinformatics, CH-1015 Lausanne, Switzerland).

LALIGN is described, see, e.g., Biro (2003) (2003) Overlapping translation of nucleic acid sequences for bioinformatics applications in Med Hypotheses. 60:654-659.

Applying One or More of the SNPs of the Present Disclosure to Datasets that Consist of Data from Early Onset Stroke Victim Subjects and Control Subjects

The novel and inventive list of SNPs that is provided by the present disclosure can be used to interrogate a dataset that consists of data from early onset stroke victims and from control human subjects. The result of this interrogation is the separation of the dataset into two sets, where the first set contains data that is at least 60% from stroke victims and less than 60% from controls, at least 70% from stroke victims and less than 30% from controls, at least 80% from stroke victims and less than 20% from controls, at least 80% from stroke victims and less than 20% from controls, at least 90% from stroke victims and less than 10% from controls, at least 95% from stroke victims and less than 5% from controls. The interrogation can be with one SNP, with two SNPs, with three SNPs, with four SNPs, with five SNPs, with six SNPs, with seven SNPs, with eight SNPs, with nine SNPs, with ten SNPs, with 11 SNPs, with 12 SNPs, with 13 SNPs, and so on. The interrogation can be conducted with all of the SNPs that are provided by the present disclosure. To summarize, the entire dataset is interrogated with a specific group of SNPs, perhaps five SNPs, and the result is the division of the dataset into two halves, a first half that is enriched in early onset stroke victims, and a second half that is enriched in control human subjects.

### Genetic and Biochemical Methods

Methods and equipment are available for genetic analysis and the identification of SNPs, for example, Homogeneous MassExtend reactions to prepare PCR reaction products for analysis by mass spectrometry (Sequenom, San Diego, Calif.), the iSelect Infinium assay (Illumina, San Diego, Calif.), TaqMan® technology and PCR System (Life Technology, Foster city, CA). For use in labeling nucleic acids, a composition that is “labeled” is detectable, either directly or indirectly, by spectroscopic, photochemical, biochemical, immunochemical, isotopic, or chemical methods. Useful labels include ³²P, ³³P, ³⁵S, ¹⁴C, ³H, ¹²⁵I, stable isotopes, epitope tags, fluorescent dyes, electron-dense reagents, substrates, or enzymes, e.g., as used in enzyme-linked immunoassays, or fluorettes (see, e.g., Rozinov and Nolan (1998) Chem. Biol. 5:713-728). The skilled artisan can determine vitamin status in a human subject by standard methods, such as microbiological assays, competitive binding assays, and the like (see, e.g., Brody (1999) Vitamins in Nutritional Biochemistry, Academic Press, San Diego, pp. 491-692).

### Genome Wide Associate Studies of Stroke

The search for stroke susceptibility genes has been discouraging; the candidate gene approach using single nucleotide polymorphisms (SNPs) in a few genes in case-control studies has had limited success. Genome-wide association studies (GWAS) have generally identified SNPs in specific genes with low odds ratios and have been underpowered statistically. Metaanalysis of these GWAS studies, involving large sample, have identified some genes, but this too has not been definitive in many cases until recently (3848 International Stroke Genetics Consortium (ISGC) 2012). GWAS and metaanalysis of GWAS approaches suffer from racial populations differences, phenotypic heterogeneity, identification of genes that may or may not play a contributing or major role in stroke, confounding effects of risk-increasing disorders, i.e. hypertension, variation between diagnosis methods, i.e. neuroimaging or clinical diagnosis, lack of differentiation based on stroke subtype, unreplicated false positive associations, true associations failing replication due to false negatives in underpowered studies, lack of positivity in one population versus another for a gene, and a bias on the microarray for common SNPs. To date, “GWAS can only explain a few percent of the apparent genetic variance contributing to common disease” (3997 Lupski, J. R. 2011). Further, selection issues for identifying relevant gene may be operative because studies are done on patients that survive stroke, which has a significant initial and early mortality (3825 Markus, H. S. 2012). Matarin concluded that “no single common gene variant exerted a major risk for stroke (377 Matarin, M. 2010).

Although these limitations argue against the validity of the common variant gene relationship to stroke pathophysiology, well designed recent studies with large populations, differentiated stroke subtypes by TOAST criteria alone or with and without early onset stroke have shown replicatable results with unique identified genes (3848 International Stroke Genetics Consortium (ISGC) 2012; 3853 Cole, J. W. 2011; 3829 Markus, H. S. 2012) (581 Matarin, M. 2008; 4039 Anderson, C. D. 2010; 382 Gschwendtner, A. 2009; 4047 Ikram, M. A. 2009; 4042 Bevan, S. 2012). These include cardioembolic stroke associations near PITX2 (4q25) and ZFHX3 (16q22.3), and large vessel stroke at a 9p21 locus (CDKN2A, DCKN2B) (3848 International Stroke Genetics Consortium (ISGC) 2012), as well as HDAC9, histone deacetylase 9, on chromosome 7p21.1 (3848 International Stroke Genetics Consortium (ISGC) 2012). GWAS has also demonstrated that chromosome 9p21.3 locus may be associated with coronary artery disease, ischemic stroke, and platelet reactivity (reviewed in (3853 Cole, J. W. 2011). In a large GWAS study, two SNPs close to the ninjurin 2 gene on chromosome 12p13 were significantly associated with stroke and ischemic stroke, in a general population with population attributable risks of 11% to 13% and 14% to 17%, respectively, and one of these SNPs in a racially segregated fashion, i.e. African Americans with ischemic stroke (reviewed in (3853 Cole, J. W. 2011). A recent international GWAS meta-analysis including a large number of cases from the Ischemic Stroke Genetic Study (younger age biased, ISGS), the Sibling with Ischemic Stroke Study (SWISS, younger age biased), and Biorepository of DNA in Stroke (BRAINS) datasets, did not demonstrate a common variant that contributed to moderate risk of ischemic stroke risk (3863 Meschia, J. F. 2011) or in stroke subtypes by TOAST criteria. The GWAS approach was inferior to using familial history of stroke for defining risk (3863 Meschia, J. F. 2011). Alternatively, in a well-defined population of Caucasian patients from the Genes Affecting Stroke Risk and Outcome Study (GASROS), a multivariate study using Bayesian analysis of SNPs that might be contributing to cardio embolic stroke in whites, identified 37 SNPs SNPs in 20 genes and intronic regions, including a homocysteine and folate acid metabolism gene, MTR (348 Ramoni, R. B. 2009).

Taken together, these observations suggest heterogeneity of genetic effects between stroke subtypes (3848 International Stroke Genetics Consortium (ISGC) 2012). Biologically, the effects of these genes and their variation, as with HDAC9, has not been established in terms of stroke pathophysiology. Further despite using strict subtype criteria, these criteria may be inaccurate and arbitrary in some cases (3848 International Stroke Genetics Consortium (ISGC) 2012). Despite significant p values, the odds ratios for these gene associations range from 1.0 to 1.58 (3848 International Stroke Genetics Consortium (ISGC) 2012). This suggested that distinct stroke subtypes versus all stroke could be used, because “genetically homogeneous stroke subtypes” may have “specific genetic risk profiles” (3836 Hacke, W. 2012). “A combination of multiple risk alleles may lead to the identification of high risk multilocus genotype patterns.” (3836 Hacke, W. 2012); the common disease, common variant hypothesis may be applicable to stroke. All of these GWAS studies and GWAS meta-analysis studies are guided by a shotgut approach that is not predicated on biological knowledge that is relevant to putative stroke physiology. Here we explore common variants in genes that may act together to increase stroke risk, which is guided by biological knowledge about pathways and their genes that could play a role in stroke etiology.

Biological Pathways with Potential Role in Stroke Pathology

The problem for prior stroke studies is that GWAS and association studies suggest association but do not validate causality. Further, the functional role of the genes and their variants identified in stroke pathophysiology is not clear. Using microarray analysis of RNA from whole blood but not brain, unique patterns of reduced or increased expression mRNA expression of certain genes or probe set patterns of expression, are seen including but not limited to inflammatory, atherogenic, and oxidative stress genes in distinct stroke subtypes, i.e. large vessel, cardioembolic, and white matter hyperintensities. Sharp and his group have focused on inflammatory genes and potential pathways in stroke pathophysiology (4008 Stamova, B. 2010; 4004 Sharp, F. R. 2011; 4005 Sharp, F. R. 2011; 365 Sharp, F. R. 2007; 4000 Jickling, G. C. 2012; 4003 Jickling, G. C. 2011).

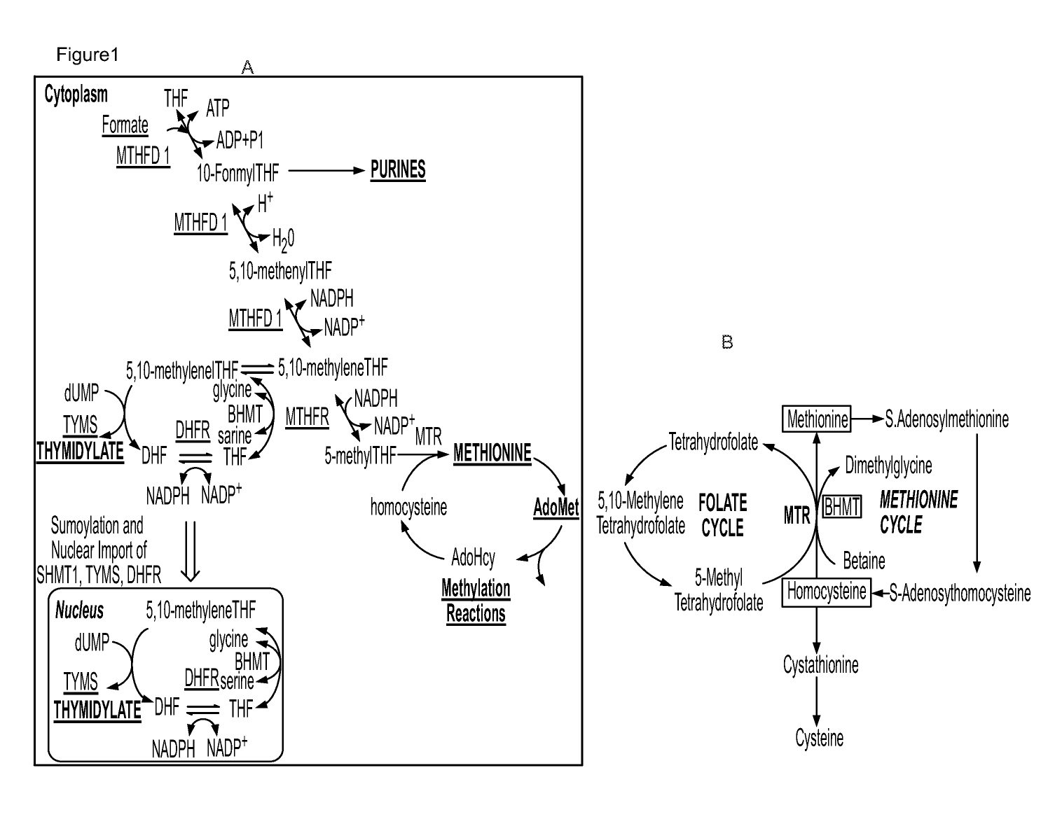

Another specific set of potential pathways for stroke risk and pathophysiology may be derived from prior biological knowledge with the 1-carbon folate dependent pathway (FIG. 1). Genes that mediate 1-carbon metabolism comprise mutations and genetic polymorphisms, for example, MTHFR C677T, MTHFR A1298C, MTR, and combinations of these with mutations and polymorphisms in folate receptors and transporters may be associated with stroke susceptibility (478 Xin, X. Y. 2009; 3882 McNulty, H. 2012; 1374 Alluri, R. V. 2005; 1456 Leclerc, D. 2007; 1193 Kim, R. J. 2003) (348 Ramoni, R. B. 2009) (1376 Arai, H. 2007; 428 Bentley, P. 2010) as well as being associated with changes in blood markers, that is, homocysteine, folate, and other B vitamins that are intermediates or co-factors in the 1-carbon folate dependent pathay (3882 McNulty, H. 2012; 3897 Wernimont, S. M. 2011). For folate pathway markers and markers of pathway dysfunction may include the primary folate derivative, 5 methyl tetrahydrofolate (5THF) (1537 Antoniades, C. 2009) and tetrahydrofolate (THF) with or without other vitamins (B12, thiamine, or riboflavin deficiency) (3882 McNulty, H. 2012; 3897 Wernimont, S. M. 2011); molecular methylation status, including DNA methylation (3897 Wernimont, S. M. 2011) and aldomet (3896 Stover, P. J. 2011; 1224 Stover, P. J. 2009). Within this 1 carbon pathway, folate and its metabolite levels and folate deficiency and disposition have multiple independent and interrelated effects; this pathway is involved with homocysteine remethylation (MTHFR, MTRR), and thereby, homocysteine levels, thymidine synthesis (thymidylate synthase) from uracil for pyrimidine synthesis for DNA, purine synthesis for DNA synthesis (SHMT), the generation of S adenosyl methionine (MTRR, MTHFR, MTR) for methylation reactions including DNA methylation, choline production and myelination (BHMT), neurotransmitter synthesis (FPGS), regulation of apoptosis and cell survival, axonal regeneration and mitochondrial function (1193 Kim, R. J. 2003) (1478 Stover, P. J. 2009; 1240 Ifergan, I. 2008; 1203 Iskandar, B. J. 2010; 1250 Chou, Y. F. 2007), lipid peroxidation, and the regulation of the production of fibrinogen and clotting factors. Reduced thymidine with increased uracil (TYMS) and reduced choline due to excessive consumption for methylation intermediates (BHMT) may be related to 1-Carbon folate metabolic dysfunction and mutations/polymorphisms in pathway genes (3896 Stover, P. J. 2011; 1478 Stover, P. J. 2009).

Elevated homocysteine may occur in 5% of the population (1385 Devos L, Chanson A, Liu Z, Ciappio E D, Parnell L D, Mason J B, Tucker K L, Crott J W 2008) and can be related to variant SNP MTHFR mutations, C667T or A1285G (1534 Yang, Q. H. 2008; 3897 Wernimont, S. M. 2011), naturally occurring MTHFR mutations (3916 Leclerc, D. 2007) (1193 Kim, R. J. 2003) or cystathione synthase (CBS) deficiency or dysfunction (1198 Testai, F. D. 2010; 1383 Brustolin S, Guigliani R, Felix T M 2010) and also can be related to specific 1 carbon folate pathway SNPs (3897 Wernimont, S. M. 2011; 3895 Wernimont, S. M. 2012), i.e. MTHFR rs180133 (C677T), MTRR, DHFR, MTHFD1, SLC19A1 (folate transporter), SLC19A3 (thiamine transporter), FOLH1 or combined MTHFR and other 1-carbon pathway variants, i.e. RFC or MS (1538 Vaughn, J. D. 2004). SLC19A3 is associated with biotin-responsive basal ganglia disease (Zhao and Goldman (2013) Mol. Aspects Med. 34:373-385). MTHFR regulates the conversion of homocysteine to methionine, mediated by FAD and folate. MTHFR C677T(rs1801133) and mutation in the coding region that converts an alanine to valine at amino acid 222 (A222V), can cause reduced MTHFR activity with elevation in homocysteine, impaired folate metabolism and altered tissue distribution, reduced 5 MTHF (a primary folate intermediate), and increased DNA uracil (3916 Leclerc, D. 2007; 3896 Stover, P. J. 2011; 1224 Stover, P. J. 2009). This emphasizes the interdependency of 1-Carbon pathway metabolites and co-factors and their levels that adds to the difficulty of risk attribution for stroke and its pathophysiology. For MTHFR GWAS studies, the actual risks identified for ischemic stroke alone had odds ratios of 1.05 to 1.44; the prevalence of these genetic abnormalities may range from 3 to 45% in the general population, that may argue for important roles in stroke pathophysiology (428 Bentley, P. 2010). The importance of MTHFR and its dysfunction and deficiency in multiple organs is argued for in animal studies (1459 Li, D. 2006; 1456 Leclerc, D. 2007).

Severe hyperhomocystinemia and homocystinuria are disorders related to dysfunction or absence of cystathione synthase; these are associated with mental retardation and severe multisystem disease (1383 Brustolin S, Guigliani R, Felix T M 2010). Elevated homocysteine has been linked to CAD, stroke, peripheral artery disease, and venous thromboembolism (1262 Ebbing, M. 2010) (442 Nakai, K. 2009; 3882 McNulty, H. 2012), as well Alzheimers disease and dementia (1372 Seshadri, S. 2006). However, the reduction in homocysteine by vitamin therapy with co-factors, i.e. folate, vitamin B12, and vitamin B6 may or may not reduce the risk of stroke or other vascular disorders (1256 Smulders, Y. M. 2010; 3882 McNulty, H. 2012) (3889 Clarke, R. 2011; 3903 Clarke, R. 2012). Re-evaluation of the role of homocysteine lowering by folate has shown a reduction in stroke risk in the Hope-2 study (4010 Saposnik, G. 2009), and a meta-analysis of RCT “showed that homocysteine lowering by folic acid reduced the risk of stroke in general by 18%, but with significantly greater reduction in those trials for longer duration (4015 Wang, X. 2007; 4013 Lee, M. 2010; 4012 Huo, Y. 2012). Increased folate status with homocysteine lowering may be preventive for stroke (3882 McNulty, H. 2012). However, recently, a very large study showed that the MTHFR association with stroke was evident only in regions of low folate with a null effect in regions of folate sufficiency (3910 Holmes, M. V. 2011). Recently, a large meta-analysis relating the TT MTHFR genotype, homocysteine, and cardiovascular disease shows that “lifelong moderate homocysteine” has little or no effect on CHD and suggest publication bias or methodological problems for previous positive results (3901 Clarke, R. 201).

Alternatively, homocysteine level may reflect on B vitamin status, particularly folate, homocysteine breakdown, and DNA methylation (3898 Jamaluddin, M. S. 2007). MTHFR C677T is associated with increased stroke and vascular risk in multiple GWAS, candidate gene, and GWAS meta-analysis studies (478 Xin, X. Y. 2009; 3882 McNulty, H. 2012; 1374 Alluri, R. V. 2005; 1456 Leclerc, D. 2007; 1193 Kim, R. J. 2003). Stroke risk is progressively increased from wild type (CC) to heterozygote (CT) to homozygote (TT) that correlates with progressively increasing levels of homocysteine, that may suggest causality (3882 McNulty, H. 2012; 3885 Mejia Mohamed, E. H. 2011). However, MTHFR dysfunction may reflect on contributing factors in addition to homocysteine, which cannot fully explain stroke pathophysiology, including other 1-carbon pathway genes and their proteins. MTHFR may confer stroke risk, but whether or not this is through homocysteine elevation, folate abnormalities, interacting vitamin deficiencies, methylation abnormalities, or other interconnected abnormalities in the 1-carbon pathway related to MTHFR or other 1-carbon genes is unclear.

The exact relationship between folate and folate metabolite levels and their tissue distribution with stroke is unclear (1244 Sanchez-Moreno, C. 2009), but folate has interdependent effects within the whole pathway, may correct homocysteine levels, and may ameliorate the effects of MTHFR deficiency. Folate oversufficiency and sufficiency may reduce stroke risk in susceptible populations, may mask or ameliorate MTHFR and/or homocysteine effects or may play a primary independent role (Petitti, personal communication) (3882 McNulty, H. 2012). MTHFR A1298C (rs1801131) is associated with folate concentration and B12 deficiency and risk for coronary artery disease (1383 Brustolin S, Guigliani R, Felix T M 2010). Folate levels, including brain folate levels, may be independently regulated by folate transporter and receptor abnormalities, FOLR1 or a (1384 Steinfeld R, Grapp M, Kraetzner R, Dreha-Kulaczewski S, Helms G, Dechent P, Wevers R, Grosso S, Gartner J 2009; 1428 Steinfeld, R. 2009) or polymorphisms, i.e FOLH1 (1385 Devos L, Chanson A, Liu Z, Ciappio E D, Parnell L D, Mason J B, Tucker K L, Crott J W 2008). Folate and homocysteine may have independent or combinatorial molecular effects, including vascular endothelial NOS regulation (1537 Antoniades, C. 2009); 1 carbon folate pathway polymorphisms may interact with vitamin sufficiency of specific B vitamins and other vitamins to increase stroke risk (3882 McNulty, H. 2012). In addition to homocysteine, within this pathway, specific polymorphisms, including non-synonymous and missense SNPs, involving but not limited to MTHFR, MTRR, GGH, RFC (reduced folate carrier), SLC9A1 (folate transporter). SLC9A3, combinations of certain pathway SNPs and other pathway genes, have been associated with homocysteine level, folate and folate intermediate levels and DNA methylation (3897 Wernimont, S. M. 2011; 1385 Devos L, Chanson A, Liu Z, Ciappio ED, Parnell L D, Mason J B, Tucker K L, Crott J W 2008); association of folate level is also seen with cardiovascular disease risk (3895 Wernimont, S. M. 2012) and stroke risk (3882 McNulty, H. 2012). Further, prior biological knowledge using the 1 carbon folate pathway has been used in large SNP studies with spina bifida (4032 Shaw, G. M. 2009) and for microRNA targeting and regulation of pathway genes (3977 Stone, N. 2011).

Folate deficiency and disposition abnormalities, independently or in association with abnormalities in 1-carbon pathway enzymes may be associated with significant neurological and other organ disorders throughout the life cycle, including spinal bifida and other neural tube defects, Retts syndrome, Down's syndrome, and cancer (1378 Biselli, P. M. 2010; 1236 Blom, H. J. 2009; 1383 Brustolin S, Guigliani R, Felix T M 2010; 1447 Ananth, C. V. 2008; 1385 Devos L, Chanson A, Liu Z, Ciappio E D, Parnell L D, Mason J B, Tucker K L, Crott J W 2008; 1473 Beaudin, A. E. 2009; 1446 Christensen, K. E. 2009; 1253 de Vogel, S. 2009; 1202 Duthie, S. J. 2010; 1440 Ramaekers, V. T. 2003; 1220 Van Guelpen, B. 2010; 1240 Ifergan, I. 2008; 1203 Iskandar, B. J. 2010) as well as stroke and cognitive dysfunction (1435 Ivanov, A. 2009; 1249 Fenech, M. 2010; 1432 Gordon, N. 2009; 1228 Kronenberg, G. 2009; 1252 Kruman, I. I. 2002; 1246 Li, L. 2008; 1450 Chan, A. 2008). Many inborn metabolic errors with varied neurological and systemic phenotypes may be related to mutations in genes and their proteins in 1-carbon pathways (1383 Brustolin S, Guigliani R, Felix T M 2010). Apart from homocystinuria and mitochondrial disorders, stroke has not been a defining feature of these disorders, but after childhood, these defects, are not routinely sought. However, combinatorial MTHFR polymorphisms and folate receptor abnormalities have been observed (1385 Devos L, Chanson A, Liu Z, Ciappio E D, Parnell L D, Mason J B, Tucker K L, Crott J W 2008). C677T MTHFR and RFC1 intron 5 A to G polymorphisms are associated with elevated homocysteine (1385 Devos L, Chanson A, Liu Z, Ciappio E D, Parnell L D, Mason J B, Tucker K L, Crott J W 2008). The importance of the 1 carbon pathway in stroke risk and pathophysiology is emphasized by MTHFR and other enzyme polymorphisms, MTR, MTRR, that show association with stroke risk in GWAS and Bayesian based studies (348 Ramoni, R. B. 2009), as well as the observations with homocysteine and folate, but definitive mechanisms have not been found.

Although C677T and A1298C polymorphisms lead to the production of a MTHFR enzyme with reduced activity, ameliorated to some degree by folate intake, neither homocysteine alone, folate alone, or both or how the MTHFR or other 1 carbon pathway genes are involved in stroke pathophysiology has been defined. The mechanisms for transcription, pre-mrna splicing, and translation for MTHFR and other critical 1-carbon folate pathway genes are complex and involve multifactorial regulation, multiple alternative pre-mrna splicing events and microRNA regulation (3947 Stone, N. 2011; 1456 Leclerc, D. 2007; 3930 Li, F. 2008; 3983 Ghosh, S. 2012) (stone). Naturally occurring mutations in MTHFR and 1 carbon folate genes may be due to abnormalities in splicing and greater than 70% of the approximately 25,000 genes in human genome produce transcripts that are alternatively spliced (3968 Hertel, K. J. 2008). 5′ promoter abnormalities and variant SNPs have been identified for MTHFR and for TYMs and BHMT that may affect mRNA and protein levels and activity (3983 Ghosh, S. 2012; 3933 Feng, Q. 2011; 4035 Li, F. 2008). Splicing mutations with functional effects have been frequently reported with MTHFR (3911 Martin, Y. N. 2006; 3916 Leclerc, D. 2007). For splicing, a gene is transcribed and a pre-mRNA is produced that has both exonic and intronic co-linear content. The pre-mRNA is then spliced to include exons. In the intron, the process of accurately and reliably producing a valid mRNA from the exons is mediated by the consensus 5′ and 3′ splice site sequences and branch point sequences in combination with multiple cis acting splicing regulatory elements; the latter include exon splice enhancers and silencers and intron splice enhancers and silencers (3969 Wang, Y. 2012; 3971 Wang, Z. 2008; 3976 Yeo, G. W. 2007; 3974 Das, D. 2007; 3973 Hertel, K. J. 2008). The latter, similar to transcriptional regulatory factors/proteins, act as binding sites for interaction with splicing regulating factors/proteins (3969 Wang, Y. 2012; 3971 Wang, Z. 2008). Splicing specificity through these sequences and their associated regulators also determines the production of different isoforms by alternative splicing (3969 Wang, Y. 2012; 3971 Wang, Z. 2008). Alternative splicing is needed to produce tissue and developmental stage specific isoforms and may be a primary site for regulation of 1-carbon folate dependent genes.

### Pathway Analysis

Attempts have been made to identify salient biological functions for the genes identified by GWAS, mRNA expression studies, preliminary exomic sequencing (3845 Cole, J. W. 2012), and meta-analysis of GWAS studies, but investigation into specific pathway dysfunction in stroke that might involve multiple interacting genes has not been done. GSEA-SNP combines SNP association analysis with pathway driven gene set analysis that uses gene expression data (3900 Holden, M. 2008). This is predicated on the concept that SNPs that may contribute to disease risk and pathophysiology have subsets of genes in key pathways or under common regulation that may be disease related by biological mechanisms (3900 Holden, M. 2008). Glossi (Gene loci set analysis) combines prior biological knowledge into “statistical analysis of genotyping data to test the association of a group of SNPs (loci-set) with complex disease phenotypes (3927 Chai, H. S. 2009). The relevance of these approaches for complex polygenic disorders, such as stroke is important. Further, pathway analysis using Pathway/SNP (3893 Dinu, V. 2007), a software application, has been used to integrate domain knowledge with statistical and data mining for high density genomic SNP disease association analysis. This approach and set of tools has been utilized to explore in the depth the association between complement pathway genes and age related macular degeneration (1365 Dinu, V. 2007) and between APOE and related genes and Alzheimer's disease (3891 Briones, N. 2012). This approach has been utilized and modified in the current study to incorporate a selected group of genes of potential pathophysiological importance for stroke in the 1 carbon folate dependent pathway to look for common SNPs and gene associations in an early onset ischemic stroke data set in a two stage process.

The purpose of this study is to use the quality young adult stroke dataset from GENEVA and multiple genes involved in folate sufficiency, i.e. reduced folate carrier gene (SC19A1), folate receptor α and β genes (FOL1), PCFT, andfolate dependent 1 carbon pathway, i.e. MTHFR, MTR, MTHD1, MTRR, DHR, TYMS, FPGS, BHMT, SHMT to look for young adult stroke risk in a two-step process. First, Plink (1271 Purcell, S. 2007) was used on these candidate genes for GWAS using multiple statistical association measures and logistic regression. SNPs with statistical significance were then selected out based on p value threshold and analyzed in Weka data mining applications (4009 Witten, I. 2011) (4019 Frank, E. 2004) to look for gene and gene polymorphism modeling in the 1 carbon folate dependent pathway. The observed genes with significant association in stroke were then evaluated using SNP position, Polyphen 2 functional analysis, and biological knowledge and bioinformatic functional analysis methodology to evaluate their potential individual and interactive role for stroke risk and stroke pathophysiology and the potential molecular mechanisms that might be involved. In order to look for significant primary genes or groups of genes, a quality stroke dataset derived from young adult stroke, as an extreme group, was used. This University of Maryland dataset, developed and selected based on age (15-49 years), sex, race, case control status by clinical and Neuroimaging diagnosis, and validated with strict quality control criteria by Hardy Weinberg principles (3921 Cheng, Y. C. 2011) was used. This dataset has been used by (3921 Cheng, Y. C. 2011) to identify genes by preselecting genes or evaluating for candidate genes, which may or may not be pathophysiologically or biologically important in stroke.

Combining strict and accepted statistical approaches to GWAS data using the Plink platform with data mining approaches has been successfully employed to identify susceptibility genes in Alzheimer's disease (3891 Briones, N. 2012). Further, the utility of identifying potentially significant disease causing genes can be enhanced by combining GWAS analysis and pathway analysis (1364 Dinu, V. 2007). Given the potential utility of these approaches to employ and take advantage of GWAS and the utility of bioinformatic and molecular functional analysis, the current paper reports on the use of a novel combination of stepwise statistical analysis, using Plink of putative critical stroke pathway genes, based on prior GWAS and molecular studies and biological knowledge and their further analysis by data mining protocols in Weka and after gene and variance selection, the use of bioinformatic techniques and databases, including TargetScan (4028 Anonymous 2012), Exon Scan (4025 Anonymous 2012), dbSNP (4026 Anonymous 2012), Gen Bank (4024 Anonymous 2012), BLAST (4029 Anonymous 2012), UCSC genomic browser (4023 Anonymous 2012), Hap Map (4027 Anonymous 2012), Haploview (4017 Anonymous 2011), and Aceview (3992 Aceview 2006) linked with biological knowledge from KEGG (4022 Anonymous 2012) and relevant literature to look at the genes identified by Plink analysis. Bioinformatics, molecular, and statistical techniques are combined to perform this analysis. Molecular defects, including inherited genetic defects in these 1-carbon pathway enzymes, including simultaneous abnormalities or variances in multiple genes, in combination with folic acid deficiency may predispose to stroke and other neurological disorders. Using this novel platform for evaluation of a publically available early onset stroke dataset, we demonstrate that in this population of young adult that SNP variants in MTHFR, TYMS, MTRR, and BHMT that effect 1-carbon folate mediated cellular methylation and other 1-carbon folate mediated or associated genes, i.e. SLC19A3 (thiamine transporter) and folate receptor R, may be associated with stroke risk. Early stroke susceptibility may be based on the function, regulation and interaction of inherited coding and non-coding variance abnormalities in the critical 1 carbon folate mediated genes and the 1 carbon folate pathway, as well as pathways and the availability of folate, its derivatives, and other vitamin co-factors. This study may provide an initial set of genes in a key pathway that may be targets for preventive and treatment intervention in stroke populations at least in United States African American and Caucasian populations.

Therefore, what is needed is a combinatorial approach of biological pathway knowledge with stroke relevance combined with an early onset stroke dataset, Plink association statistics, logistic regression, and multilocus classifiers can be used to identify novel common gene variants in a polygenic complex disorder, such as stroke and a preliminary screen to identify genes for further evaluated of functionally and biological significance and relevance. Variants in multiple genes in the 1-carbon folate derived pathway may act alone or in combination to increase stroke risk and enhancement or silencing of alternative splicing within exons and introns in methylation economy enzymes/genes may be a common mechanism, at least in this pathway for stroke pathophysiology and risk. Novel, previously unreported SNPs for MTHFR, BHMT, MTRR, TYMS, that are primary regulators of methylation and homocysteine metabolism were identified with a potential useful platform that employed an integrated approach with focused data extraction from a large dataset, its use in Plink, extraction and processing for Weka analysis for combined GWAS and data mining followed by bioinformatic and molecular functional analysis.

### Detailed Descriptions of the Figures

FIG. 1. FIG. 1A discloses folate-mediated 1-carbon metabolism. FIG. 1B shows BHMT and the folate and methionine cycles. Folate and its major metabolite, 5 methyl tetrahydrofolic acid (5 methyl THF), derived from dietary sources enters at the intestine and through cells by the reduced folate carriers and proton coupled folate transporter (PCFT). The folate receptors α and β are folate binding cellular receptors that use endocytosis to transport folate into cells. 5 Methyl THF is a primary component of the 1-carbon folate pathway and along with other folate derivatives provides carbons for purine, methylation, and thymidine synthesis. The 1 carbon folate dependent pathway comprises 3 interdependent biosynthetic pathways. These include: 1) de novo purine biosynthesis that involves 10-formyl THF, requiring formate incorporation into 10 formyl THF by MTHFD1, in an ATP dependent reaction involving other enzymes (SHMT); 2) de novo thymidylate biosynthesis (TYMS), which may also occur in the nucleus; and 3) the remethylation of homocysteine to methionine (MTHFR, MTR, MTRR).

MTHFR has a primary role in generating carbons for methylation as part of the s-adenosyl methionine cycle, and MTHFR's function influences thymidine synthesis, purine synthesis, DNA efficacy, and also normal operation of clotting pathways. The carbons for methylation are derived from methionine, which is also an essential amino acid for protein synthesis and key component of the 1 carbon folate dependent pathway.

Methylenetetrahydrofolate reductase (MTHFR) reduces 5, 10-methylenetetrahydrofolate (5, 10 Methyl THF) to 5-methyltetrahydrofolate, a NAD (P)-dependent enzyme, that is phylogenetically conserved. 5-methyltetrahydrofolate is utilized by methionine synthase (MTR) to convert homocysteine to methionine, but this enzyme can become inactive. Methionine synthase reductase (MTRR) regenerates a functional methionine synthase via reductive methylation. The active site for the protein MTHFR binds FAD and folic acid facilitates that binding and prevents dissociation, which may account for the protective actions of folates in MTHFR dysfunction. Certain novel MTHFR and MTRR polymorphisms are associated with stroke in this study. For TYMS, by reductive methylation, deoxyuridine monophosphate (dUMP) and 5,10-methyl THF are used to form dTMP, which is only source for production of dTMP and the balance of DNA nucleotide precursors. 5 10 MTHF is competed for by thymidylate synthase and by MTHFR for conversion to 5 MTHF. The MTHFR C677T genotype (rs1801133) is associated with a 34% lower DNA uracil count, because TYMS may be favored. Folate depletion reduces uridine conversion to thymidine and results in an accumulation of uracil, misincorporation of uracil into DNA, and later, increased DNA breakage, which may predispose to cancer. Betaine-homocysteine methyltransferase (Panel B) (BHMT) catalyzes the conversion of betaine and homocysteine to dimethylglycine and methionine, and thus remethylation of homocysteine. BHMT may catalyze as much as 50% of homocysteine remethylation. Choline is the source for betaine and the alternative and primary contributor in states of deficiency, such as MTHFR dysfunction. Novel polymorphisms in MTHFR, MTRR, and TYMS are statistically associated with stroke in the current study. 5 MTHF may be reduced due to dietary deficiency, abnormal function of the folate receptors and transporters, or dysfunction of MTHFR. The deficiency of 5 methyl THF can result from a partial or complete deficiency of MTHFR, but also changes in distribution of folate to specific cells and organs. Levels of 5 methyl THF are markedly reduced in brain (13% versus normal mice) and liver in mice that are deficient in MTHFR, as is total folate that is circulating. Homocysteine elevation may be due to dysfunction of cystathinoine B synthase, the cause for hyperhomocystinemia and homocystinuria., MTHFR dysfunction. MTHFR leads to a reduction in folate and 5 methyl THF. Disruption of the 1-carbon folate dependent pathways by alterations in critical enzyme activity or cellular localization or co-factor deficiency or substrate excess or deficiency may lead to abnormalities in pathway functions, including reduced methylation, reduced S-adenosyl methionine, reduced protein synthesis, reduced thymidine and increased uracil with abnormal DNA synthesis, reduced choline and subsequently reduced neurotransmitter and myelination, elevated homocysteine, and reduced and abnormally localized folate.

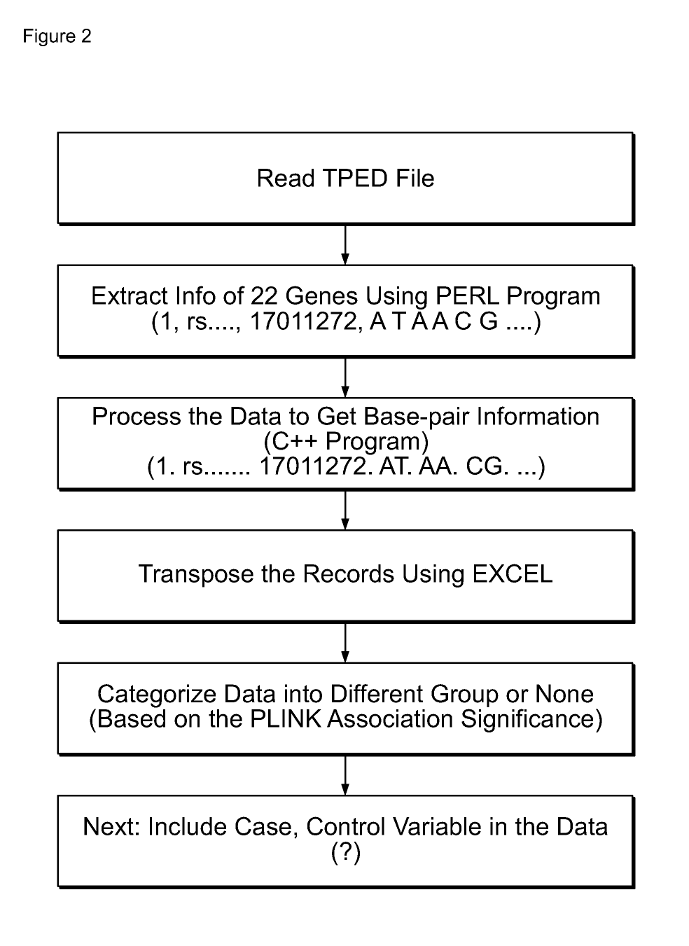

FIG. 2 illustrates the steps of reading TPED file, extracting information, processing the data, transposing the records, categorizing the data, and including case, control variables in the data.

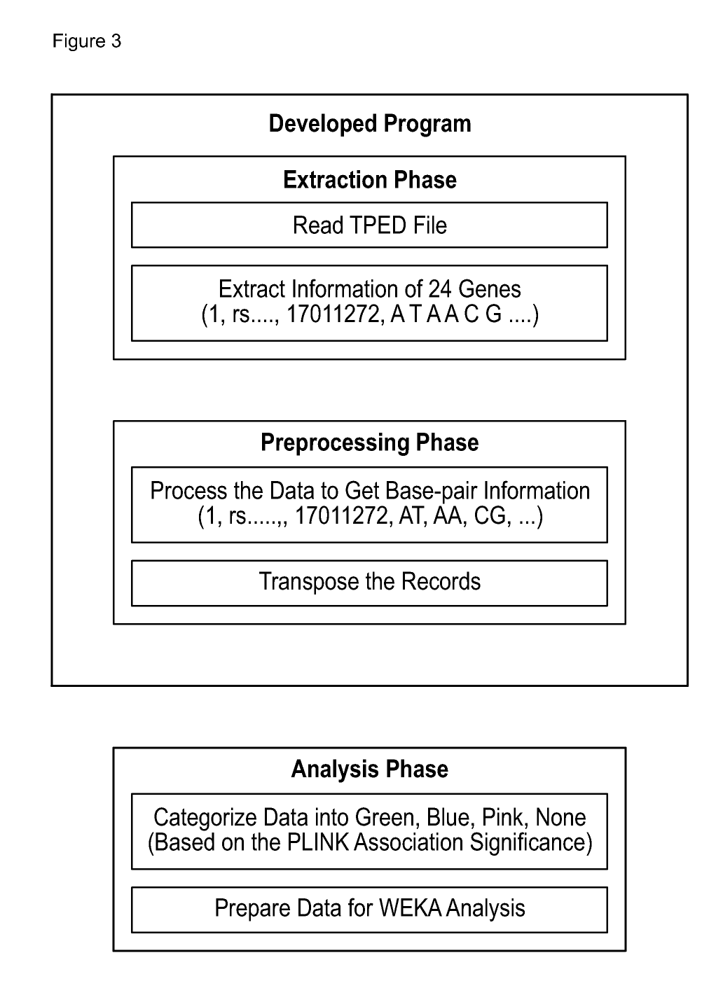

FIG. 3 shows the platform for Plink and Weka analysis, and illustrates the developed program, which comprises extraction phase and preprocessing phase, and analysis phase.

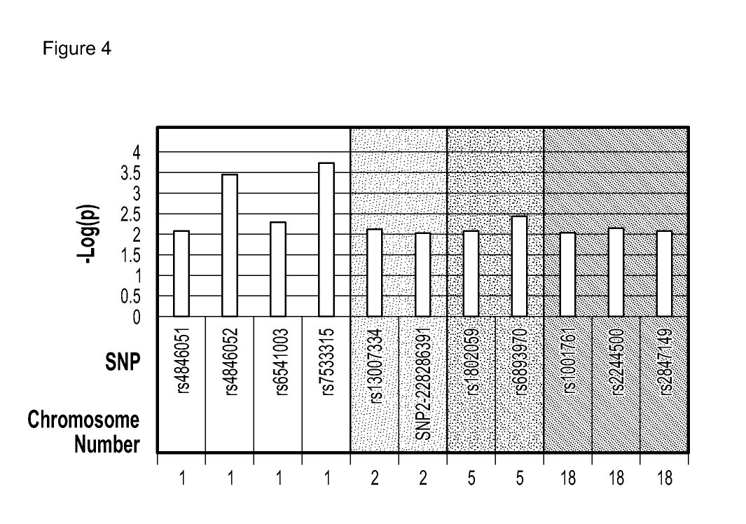

FIG. 4 show P values for eleven (11) SNPs. These SNPs were identified by Early Age Onset Stroke Association Algorithm.

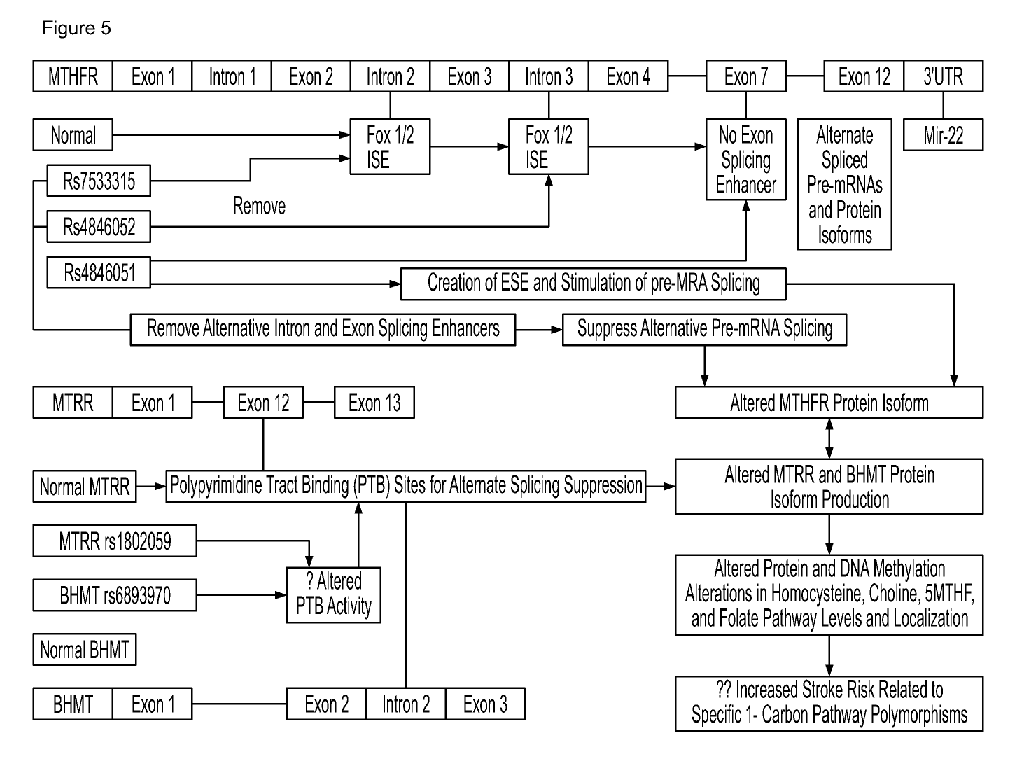

FIG. 5 shows map of MTHFR, revealing locations of exons, introns, and SNPs. Information on mRNA splicing is also given.

### EXAMPLES

The study utilized the GENEVA (Early Onset Stroke Dataset) that was obtained from the database of genotypes and phenotypes (dbGaP) database (4031 Anonymous 2012). See world wide web at the address, ncbi.nlm.nih.gov/gap.

The present disclosure shows genes and identifies one or more Single Nucleotide Polymorphisms (SNPs) within each gene. The table also discloses the correlation, in terms of P value, between the existence of the SNP as determined by genetic analysis of the sample of human study subjects, and early stroke. The smaller the P value, the stronger the association between the existence of that SNP in any given human subject, and risk for stroke. The table also discloses the location of the SNP, that is, whether the SNP is located in an intron or in an exon for each of the genes.

The present disclosure provides one SNP, or two SNPs, three SNPs, four SNPs, five SNPs, six SNPs, and the like, that were identified by the methods of the present analysis, and selected from all of the detected SNPs from a particular gene. The SNPs were selected from all of the identified SNPs of a particular gene, for example, from 25-30 SNPs, 30-35 SNPs, 35-40 SNPs, 40-45 SNPs, 45-50 SNPs, 50-60 SNPs, 60-70 SNPs, 70-80 SNPs, 80-90 SNPs, 90-100 SNPs, 100-150 SNPs, 120-170 SNPs, 140-190 SNPs, 160-210 SNPs, 180-230 SNPs, 200-250 SNPs, or more. The SNPs were selected from all of the identified SNPs of a particular gene, for example, from a gene having over 25 SNPs, over 30 SNPs, over 40 SNPs, over 50 SNPs, over 60 SNPs, over 70 SNPs, over 80 SNPs, over 90 SNPs, over 100 SNPs, over 110 SNPs, over 120 SNPs, over 130 SNPs, over 140 SNPs, over 150 SNPs, over 160 SNPs, over 170 SNPs, over 180 SNPs, over 190 SNPs, over 200 SNPs, and so on.

### Influence of SNP on Splicing

The following concerns the issue of whether a SNP occurring in an intron can influence the primary sequence of the expressed polypeptide. Where the SNP results in a change in splicing site, the result can be a change in the primary sequence. In contrast, where the SNP occurs in a part of an intron that has no result on the splicing site, the SNP is not likely to have any influence on the primary sequence. The Single Nucleotide Polymorphism in MTRR that is rs1802059, which occurs in an exon, likely results in a splicing abnormality. All of the SNPs that occur within introns, with the exception of the SNP in MTHFR that is rs6541003, likely result in splicing abnormalities. Thus, where a SNP occurs in an exon, this is likely to have a direct influence on the polynucleotide sequence, and thus provides an avenue for a mechanistic connection between the SNP, a change in polypeptide sequence, and the consequent increased risk for early stroke. Where a SNP occurs in an intron, and where this SNP results in a splicing abnormality that creates an abnormal polypeptide sequence, this can also explain the consequent increased risk for early stroke. Guidance is available for determining the influence of SNPs on splicing, and for determining the sequence of splicing sites (see, e.g., Mucaki et al (2013) Hum. Mutat. 34:557-565; Houdayer (2011) Methods Mol. Biol. 760:269-281; Sonnenburg et al (2007) BMC Bioinformatics. 8 Suppl. 10:S7; Li et al (2012) Genetics Mol. Res. 11:3432-3451). The SNPs in thymidylate synthase gene (rs2847149, rs2244500, rs1001761) are predicted to not influence and to not interfere with splicing.

SNP Embodiments that Correlate the Detection of a Given SNP with Risk for Early Stage Stroke

The present disclosure provides a system, and related methods, that make use only of SNPs from MTHFR. Also, the disclosure provides a system, and related methods, that make use only of SNPs from MTRR. Furthermore, the disclosure provides a system, and related methods, that make use only of one or more SNPs from BHMT. Moreover, the disclosure provides a system, and related methods, that make use only of one or more SNPs from BHMT. Also, the disclosure provides a system, and related methods, that make use only of one or more SNPs from SCL19A3. Also provided, is a system and related methods, that make use only of one or more SNPs from TYMS. Further provided is a system and related methods that make use only of one or more SNPs from FOLR2.

In exclusionary embodiments, the present disclosure provides a system, and related methods, that makes use of one or more SNPs, but does not make use of the SNP that is rs7533315. Also, the disclosure provides a system, and related methods, that makes use of one or more SNPs, but does not make use of rs4846052. Also, the disclosure provides a system, and related methods, that makes use of one or more SNPs, but does not make use of rs6541003, or that does not make use of rs6541003, or that does not make use of rs4846051, or that does not make use of rs1801133, or that does not make use of rs1802059, or that does not make use of rs6893970, or that does not make use of SNP2-228286391, or that does not make use of rs13007334, or that does not make use of rs1001761, or that does not make use of rs2847149, or that does not make use of rs2244500, or that does not make use of rs229844. In other exclusionary embodiments, the disclosure provides a system, and related methods, that excludes use of two of the SNPs from Table 7, or that excludes three of the SNPs from Table 7, or that excludes four, five, six, seven, eight, nine, ten, eleven, or twelve of the SNPs from Table 7.

The present disclosure, in some embodiments, can encompass a system, and related methods, where all SNPs reside in introns, where all SNPs reside in exons, or where intronal SNPs and exonal SNPs are both represented and used in the system and methods. In some embodiments, the system and related methods use SNPs only from MTHFR and not from any other genes, only from MTRR and not from any other genes, only from BHMT and not from any other genes, only from SCL19A3 and not from any other genes, only from TYMS and not from any other genes, only from FOLR2 and not from any other genes. In other embodiments, the system and methods use SNPs from only two of the genes in Table 7, from only three of the genes in Table 7, from only four of the genes in Table 7, from only five of the genes in Table 7, and so on. The next table, that is, Table 8, provides data that is similar to that in Table 7.

Systems and Methods that have a Requirement for Abnormal Splicing

The system and methods of the present disclosure, in some embodiments, interrogates a human subject with only SNPs that are associated with abnormal splicing. In other embodiments, the system and methods of the present disclosure, interrogates the human subject with a set of SNPs, where only one of the SNPs, where only two of the SNPs, where only three of the SNPs, where only four of the SNPs, where only five of the SNPs, where only six of the SNPs, where only seven of the SNPs, where only eight of the SNPs, where only nine of the SNPs, where only ten of the SNPs, where only 11 of the SNPs, where only 12 of the SNPs, or where all of the SNPs is associated with abnormal splicing.

The system and methods of the present disclosure, in some embodiments, interrogates a human subject with only SNPs that are associated with abnormal splicing. In other embodiments, the system and methods of the present disclosure, interrogates the human subject with a set of SNPs, where at least one of the SNPs, where at least two of the SNPs, where at least three of the SNPs, where at least four of the SNPs, where at least five of the SNPs, where at least six of the SNPs, where at least seven of the SNPs, where at least eight of the SNPs, where only nine of the SNPs, where at least ten of the SNPs, where at least 11 of the SNPs, where at least 12 of the SNPs, or where all of the SNPs is associated with abnormal splicing.

TABLE 7
(first part of two parts)

| | | | Lowest | Pearson P | 35 |
|---|---|---|---|---|---|
| Gene | Chromosome | SNP | p < 0.001 | chi square | OR |
| MTHFR | 1 | rs7533315 | 0.0001984 | 0.0008937 | 0.78 |
| MTHFR | 1 | rs4846052 | 0.0003744 | 0.0003744 | 1.2 |
| MTHFR | 1 | rs6541003 | 0.005417 | 0.008 | 0.84 |
| MTHFR | 1 | rs4846051 | 0.008874 | 0.0088 | 0.77 |
| MTHFR | 1 | rs1801133* | 0.01491 | 0.01491 | 1.14 |
| | | | | (NS) | |
| MTRR | 5 | rs1802059 | 0.009128 | NS | — |
| BHMT | 5 | rs6893970 | 0.003818 | 0.011 | 1.33 |
| | | | | (NS) | |
| SLC19A3 | 2 | SNP2- | 0.009701 | 0.0097 | 0.84 |
| | | 228286391 | | | |
| SLC19A3 | 2 | rs13007334 | 0.007722 | 0.143 | 1.105 |
| | | | | (NS) | |
| TYMS | 18 | rs1001761 | 0.009726 | 0.00297 | 1.16 |
| | | | | (NS) | |
| TYMS | 18 | rs2847149 | 0.008865 | 0.0123 | 1.17 |
| | | | | (NS) | |
| TYMS | 18 | rs2244500 | 0.007641 | 0.17 | 1.174 |
| | | | | (NS) | |
| FOLR2 | 11 | rs229844 | 0.002996 | NS | — |

(second part of two parts)

| | | | | Alternative | |
|---|---|---|---|---|---|
| | | | Haplotype | splice | |
| SNP | CI (95) | model | block | abnormality | location |
| rs7533315 | 0.67-0.90 | recessive | 2 (small) | Loss of ISS | intron |
| rs4846052 | 1.11-1.45 | dominant | 1 | Loss of ISS | intron |
| rs6541003 | 0.74-0.96 | dominant | 1 | no | intron |
| rs4846051 | 0.10-0.63 | allelic | No block | Gain ESS | exon |
| rs1801133* | 0.98-1.33 | dominant | 1 | Abn. protein | exon |
| rs1802059 | | dominant | 3 | PBT change | exon |
| rs6893970 | 1.07-0.96 | dominant | 3 | PBT change | intron |
| SNP2- | 0.74-0.066 | NA | Un-known | | |
| 228286391 | | | | | |
| rs13007334 | 0.97-1.26 | recessive | No block | GGG in both | intron |
| rs1001761 | 1.02-1.33 | dominant | 1 | no | Intron |
| rs2847149 | 1.02-1.33 | dominant | 1 | no | intron |
| rs2244500 | 1.03-1.34 | dominant | 1 | no | intron |
| rs229844 | | dominant | No blocks | PBT creation | Intron |
| | | | for any | | |
| | | | SNPs | | |

*rs1801133 (C677T), pathological SNP, previously associated with stroke;
OR = odds ratio;
CI = Confidence Interval;
ISS = Intron Splicing Silencer;
ESS = Exon Splicing Silencer;
PBT = polypyrimidine binding tract.

TABLE 8

| Chromosome | | | |
|---|---|---|---|
| number | SNP | −Log(p) | P value |
| 1 | rs4846051 | 2.051881 | 0.008874 |
| 1 | rs4846052 | 3.426664 | 0.000374 |
| 1 | rs6541003 | 2.266241 | 0.005417 |
| 1 | rs7533315 | 3.702458 | 0.000198 |
| 2 | rs13007334 | 2.11227 | 0.007722 |
| 2 | SNP2- | 2.013183 | 0.009701 |
| | 228286391 | | |
| 5 | rs1802059 | 2.039624 | 0.009128 |
| 5 | rs6893970 | 2.418164 | 0.003818 |
| 18 | rs1001761 | 2.012066 | 0.009726 |
| 18 | rs2244500 | 2.11685 | 0.007641 |
| 18 | rs2847149 | 2.052321 | 0.008865 |

Alleles

The following table discloses alleles of the SNPs of the present disclosure, that is, which SNPs are homogenous and which are heterologeneous. F_A Frequency of this allele in cases. F_U Frequency of this allele in controls.

TABLE 9
Homogeneous and heterogenous SNP alleles

| CHR | SNP | BP | A1 | F_A | F_U | A2 | CHISQ | P | OR | SE | L95 | U95 |
|---|---|---|---|---|---|---|---|---|---|---|---|---|
| 1 | | | | | | | | | | | | |
| 1 | rs4846051 | 11777044 | G | 0.1098 | 0.1384 | A | 6.848 | 0.008874 | 0.7677 | 0.1012 | 0.6296 | 0.9362 |
| 1 | rs1801131 | 11777063 | C | 0.2629 | 0.2678 | A | 0.11 | 0.7401 | 0.9753 | 0.07524 | 0.8416 | 1.13 |
| 1 | rs6541003 | 11778454 | G | 0.4499 | 0.4938 | A | 6.985 | 0.008219 | 0.8386 | 0.06661 | 0.736 | 0.9556 |
| 1 | rs1801133 | 11778965 | T | 0.254 | 0.2302 | C | 2.802 | 0.09415 | 1.139 | 0.07767 | 0.978 | 1.326 |
| 1 | rs4846052 | 11780538 | C | 0.451 | 0.3927 | T | 12.66 | 0.0003744 | 1.271 | 0.0674 | 1.114 | 1.45 |
| 1 | rs7533315 | 11783270 | T | 0.2433 | 0.2921 | C | 11.04 | 0.0008937 | 0.7791 | 0.07521 | 0.6724 | 0.9029 |
| 2 | rs13007334 | 228283251 | T | 0.412 | 0.3881 | C | 2.145 | 0.1431 | 1.105 | 0.06788 | 0.9669 | 1.262 |
| 2 | SNP2-228286391 | 228286391 | T | 0.4055 | 0.448 | C | 6.689 | 0.009701 | 0.8404 | 0.06726 | 0.7366 | 0.9588 |
| 5 | rs1802059 | 7950319 | A | 0.3256 | 0.2927 | G | 4.609 | 0.03181 | 1.167 | 0.07197 | 1.013 | 1.344 |
| 5 | rs6893970 | 78446453 | A | 0.1098 | 0.08475 | G | 6.46 | 0.01104 | 1.332 | 0.1131 | 1.067 | 1.663 |
| 18 | rs2244500 | 651005 | T | 0.4332 | 0.3944 | C | 5.632 | 0.01763 | 1.174 | 0.06753 | 1.028 | 1.34 |
| 18 | rs1001761 | 652103 | C | 0.4333 | 0.396 | T | 5.171 | 0.02297 | 1.166 | 0.06748 | 1.021 | 1.331 |
| 18 | rs2847149 | 656371 | G | 0.4337 | 0.396 | A | 5.298 | 0.02135 | 1.168 | 0.0675 | 1.023 | 1.333 |

A Human Subject can be Interrogated for the Presence or Absence of One Particular SNP, or the Presence or Absence of a Plurality of SNPs, Before Arriving at a go/No go Decision

Table 10A to Table 10J, disclose the number of SNPs that can be used in interrogating genomic data of a given human subject. Interrogation can be with one SNP, with two SNPs, or with a plurality of SNPs, as indicated in the tables. The indicated SNPs can be limited to only those that are disclosed in the indicated combination, or the indicates SNPs can comprise SNPs in the indicated combination plus one or more SNPs in other genes that mediate 1-carbon metabolism, as well as SNPs in genes that do not mediate 1-carbon metabolism. Interrogation can be with a query that comprises the indicated SNP or, alternatively, the interrogation can be with a query that consists only of the indicated SNP or SNPs.

TABLE 10A
Test involving only one of the following SNPs or alternatively, a test
involving only one of the following SNPs plus one or more SNPs
that are not listed in
this table.

| Test | SNP |
|---|---|
| 1 | rs7533315 |
| 2 | rs4846052 |
| 3 | rs6541003 |
| 4 | rs4846051 |
| 5 | rs1801133* |
| 6 | rs1802059 |
| 7 | rs6893970 |
| 8 | SNP2-228286391 |
| 9 | rs13007334 |
| 10 | rs1001761 |
| 11 | rs2847149 |
| 12 | rs2244500 |
| 13 | rs229844 |

TABLE 10B
rs7533315 family. Tests involving only two SNPs or alternatively,
a test involving two of the following listed SNPs plus one or more
SNPs that are not listed in this table, or a test involving the
indicated two SNPs plus one or more SNP additional
SNPs that are listed in Table 8A.

| Test | SNPs |
|---|---|
| 21 | rs7533315 + rs4846052 |
| 22 | rs7533315 + rs6541003 |
| 23 | rs7533315 + rs4846051 |
| 24 | rs7533315 + rs1801133* |
| 25 | rs7533315 + rs1802059 |
| 26 | rs7533315 + rs6893970 |
| 27 | rs7533315 + SNP2-228286391 |
| 28 | rs7533315 + rs13007334 |
| 29 | rs7533315 + rs1001761 |
| 30 | rs7533315 + rs2847149 |
| 31 | rs7533315 + rs2244500 |
| 32 | rs7533315 + rs229844 |

TABLE 10C

| | |
|---|---|
| Test | SNPs |

rs4846052 family. Test involving only two SNPs or alternatively, a test
involving only two SNPs plus one or more SNPs that are not listed
in this table, or a test involving the indicated two SNPs plus one or
more SNPs that are listed in Table 8A.

| | |
|---|---|
| 41 | rs4846052 + rs6541003 |
| 42 | rs4846052 + rs4846051 |
| 43 | rs4846052 + rs1801133* |
| 44 | rs4846052 + rs1802059 |
| 45 | rs4846052 + rs6893970 |
| 46 | rs4846052 + SNP2-228286391 |
| 47 | rs4846052 + rs13007334 |
| 48 | rs4846052 + rs1001761 |
| 49 | rs4846052 + rs2847149 |
| 50 | rs4846052 + rs2244500 |
| 51 | rs4846052 + rs229844 |

rs6541003 family. Tests involving only two SNPs or alternatively, a
test involving two of the following listed SNPs plus one or more
SNPs that are not listed in this table, or a test involving the indicated
two SNPs plus one or more SNP additional SNPs that are listed
in Table 8A.

| | |
|---|---|
| 61 | rs6541003 + rs4846051 |
| 62 | rs6541003 + rs1801133* |
| 63 | rs6541003 + rs1802059 |
| 64 | rs6541003 + rs6893970 |
| 65 | rs6541003 + SNP2-228286391 |
| 66 | rs6541003 + rs13007334 |
| 67 | rs6541003 + rs1001761 |
| 68 | rs6541003 + rs2847149 |
| 69 | rs6541003 + rs2244500 |
| 70 | rs6541003 + rs229844 |

TABLE 10D
rs4846051 family. Tests involving only two SNPs or alternatively,
a test involving two of the following listed SNPs plus one or more
SNPs that are not listed in this table, or a test involving the
indicated two SNPs plus one or more SNP additional SNPs
that are listed in Table 8A.

| Test | SNPs |
|---|---|
| 71 | rs4846051 + rs1801133* |
| 72 | rs4846051 + rs1802059 |
| 73 | rs4846051 + rs6893970 |
| 74 | rs4846051 + SNP2-228286391 |
| 75 | rs4846051 + rs13007334 |
| 76 | rs4846051 + rs1001761 |
| 77 | rs4846051 + rs2847149 |
| 78 | rs4846051 + rs2244500 |
| 79 | rs4846051 + rs229844 |

TABLE 10E
rs1801133* family. Tests involving only two SNPs or alternatively, a
test involving two of the following listed SNPs plus one or more
SNPs that are not listed in this table, or a test involving the
indicated two SNPs plus one or more SNP additional SNPs
that are listed in Table 8A.

| Test | SNPs |
|---|---|
| 81 | rs1801133* + rs1802059 |
| 82 | rs1801133* + rs6893970 |
| 83 | rs1801133* + SNP2-228286391 |
| 84 | rs1801133* + rs13007334 |
| 85 | rs1801133* + rs1001761 |
| 86 | rs1801133* + rs2847149 |
| 87 | rs1801133* + rs2244500 |
| 88 | rs1801133* + rs229844 |

TABLE 10F
rs1802059 family. Tests involving only two SNPs or alternatively, a
test involving two of the following listed SNPs plus one or more
SNPs that are not listed in this table, or a test involving the
indicated two SNPs plus one or more SNP additional SNPs
that are listed in Table 8A.

| Test | SNPs |
|---|---|
| 91 | rs1802059 + rs6893970 |
| 92 | rs1802059 + SNP2-228286391 |
| 93 | rs1802059 + rs13007334 |
| 94 | rs1802059 + rs1001761 |
| 95 | rs1802059 + rs2847149 |
| 96 | rs1802059 + rs2244500 |
| 97 | rs1802059 + rs229844 |

TABLE 10G
rs6893970 family. Tests involving only two SNPs or alternatively, a
test involving two of the following listed SNPs plus one or more
SNPs that are not listed in this table, or a test involving the indicated
two SNPs plus one or more SNP additional SNPs that are listed
in Table 8A.

| Test | SNPs |
|---|---|
| 101 | rs6893970 + SNP2-228286391 |
| 102 | rs6893970 + rs13007334 |
| 103 | rs6893970 + rs1001761 |
| 104 | rs6893970 + rs2847149 |
| 105 | rs6893970 + rs2244500 |
| 106 | rs6893970 + rs229844 |

TABLE 10F
SNP2-228286391 family. Tests involving only two SNPs or
alternatively, a test involving two of the following listed SNPs plus
one or more SNPs that are not listed in this table, or a test
involving the indicated two SNPs plus one or more SNP
additional SNPs that are listed in Table 8A.

| Test | SNPs |
|---|---|
| 111 | SNP2-228286391 + rs13007334 |
| 112 | SNP2-228286391 + rs1001761 |
| 113 | SNP2-228286391 + rs2847149 |
| 114 | SNP2-228286391 + rs2244500 |
| 115 | SNP2-228286391 + rs229844 |

TABLE 8G
rs13007334 family. Tests involving only two SNPs or alternatively, a
test involving two of the following listed SNPs plus one or more
SNPs that are not listed in this table, or a test involving the indicated
two SNPs plus one or more SNP additional SNPs that are listed in
Table 8A.

| Test | SNPs |
|---|---|
| 121 | rs13007334 + rs1001761 |
| 122 | rs13007334 + rs2847149 |
| 123 | rs13007334 + rs2244500 |
| 124 | rs13007334 + rs229844 |

TABLE 8H
rs1001761 family. Tests involving only two SNPs or alternatively, a
test involving two of the following listed SNPs plus one or more SNPs
that are not listed in this table, or a test involving the indicated two
SNPs plus one or more SNP additional SNPs that are listed in
Table 8A.

| Test | SNPs |
|---|---|
| 131 | rs1001761 + rs2847149 |
| 132 | rs1001761 + rs2244500 |
| 133 | rs1001761 + rs229844 |

TABLE 10I
rs2847149 family. Tests involving only two SNPs or alternatively, a
test involving two of the following listed SNPs plus one or more SNPs
that are not listed in this table, or a test involving the indicated two
SNPs plus one or more SNP additional SNPs that are listed in
Table 8A.

| Test | SNPs |
|---|---|
| 141 | rs2847149 + rs2244500 |
| 142 | rs2847149 + rs229844 |

TABLE 10J
rs2244500 family. Tests involving only two SNPs or alternatively,
a test involving two of the following listed SNPs plus one or more
SNPs that are not listed in this table, or a test involving the indicated
two SNPs plus one or more SNP additional SNPs that are listed in
Table 8A.

| Test | SNPs |
|---|---|
| 151 | rs2244500 + rs229844 |

Step of Determining Risk can be Integrated with One or More Steps of Diagnosis and Treatment

In a non-limiting embodiment, the system and method accepts SNP data from a human subject, responds by indicating if the SNP data matches one or more SNPs from Table 7 (with the exception of rs1801133, which has a P value that is relatively insignificant). Where the human subject's SNP data matches one or more of the SNPs from Table 7, the meaning is that the subject is at increased risk for early stroke. What is integrated with the above step, is one or more subsequent steps. The one or more subsequent steps can be processing the subject to a neurological exam, processing the human subject with a medical and clinical history, administering one or more of vitamin B12, folate, vitamin B6, anti-hypertensive drug, LDL-cholesterol lowering drug, HDL-elevating drug, to the human subject. The human subject can also be processed by exposing to ultrasound vibrations for imaging. The processing by imaging that assess stroke can encompass contacting the subject with ultrasound vibrations, contacting a transducer to the subject's neck, contacting the subject's carotid artery with Doppler ultrasound, and the like. Steps that can be integrated into the system and method of the present disclosure can also include one or more of, neurological examination, carotid Doppler examination, electrocardiograpm, echocardiogram, transcranial Doppler, magnetic resonance of the brain, computed tomography examination of the brain, MR or CT angiography of the brain. Integrated steps can include one or more of treatment of hypertension, obesity, hyperlipidemia, diabetes, atrial fibrillation. Treatments for hypertension include, e.g., thiazide diuretic, alpha-blocker, beta-blocker, calcium channel blocker, dihydropyridine calcium channel blocker, angiotensin-converting enzyme (ACE) inhibitor, or angiotensin receptor blocker (ARB) (see, e.g., Stern (2013) J. Clinical Hypertension. 15:748-751).

The present disclosure encompasses systems and methods that involve non-human subjects, including primates, veterinary subjects, animals of agricultural importance, experimental animals, and the like.

The present system and method encompasses steps that assess risk for early stroke, steps that diagnose early stroke, and steps that reduce risk for early stroke, e.g., that reduce risk by administering one or more of vitamin B12, folate, or vitamin B6. The present system and method can exclude steps that assess risk for late stroke, steps that are configured to detect late stroke, or steps that reduce risk of late stroke. Subgroups of late stroke that are unique to late stroke and that are not shared with early stroke include, for example, stroke due to defective amyloid protein (see, e.g., Rostagno et al (2010) Cell Mol. Life Sci. 67:581-600; Smith and Greenberg (2009) Stroke. 40:2601-2606). The present disclosure can exclude one or more of systems, reagents, instrumentation, methods of diagnosis, that relate specifically to ischemic stroke. Moreover, the present disclosure can exclude one or more of systems, reagents, instrumentation, methods of diagnosis, that relate to homogeneous populations, for example, populations that are substantially Italian ethnicity, or substantially Chinese ethnicity. In a non-limiting embodiment, the present disclosure provides systems, and related methods, relating to the detection, discovery, and use in an algorithm, of data only from human populations that are heterogeneous. Also, the present disclosure provides systems, and related methods, relating to one or more of the diagnosis, administration of pharmaceuticals, administration of ultrasound, and treatment, human populations that are heterogeneous. Examples of stroke studies involving ethnically homogeneous populations include a study of Chinese Han population (Zhao et al (2012) J. Neuroinflammation. 9:162 (8 pages).

Without limitation, “heterogenous population” can refer to a population that is, e.g., 5-15% African ethnicity (or African American) and at least 50% Caucasian, 10-20% African ethnicity (or African American) and at least 50% Caucasian, 15-25% African ethnicity (or African American) and at least 50% Caucasian, 20-30% African ethnicity (or African American) and at least 50% Caucasian, 25-35% African ethnicity (or African American) and at least 50% Caucasian, 30-40% African ethnicity (or African American) and at least 50% Caucasian, and so on.

Without limitation, “heterogenous population” can refer to a population that is, e.g., 5-15% African ethnicity (or African American) and at least 40% Caucasian, 10-20% African ethnicity (or African American) and at least 40% Caucasian, 15-25% African ethnicity (or African American) and at least 40% Caucasian, 20-30% African ethnicity (or African American) and at least 40% Caucasian, 25-35% African ethnicity (or African American) and at least 40% Caucasian, 30-40% African ethnicity (or African American) and at least 40% Caucasian, and so on.

Without limitation, “heterogenous population” can refer to a population that is, e.g., 5-15% African ethnicity (or African American) and at least 30% Caucasian, 10-20% African ethnicity (or African American) and at least 30% Caucasian, 15-25% African ethnicity (or African American) and at least 30% Caucasian, 20-30% African ethnicity (or African American) and at least 30% Caucasian, 25-35% African ethnicity (or African American) and at least 30% Caucasian, 30-40% African ethnicity (or African American) and at least 30% Caucasian, and so on.

“Heterogenous population” can also refer to a population that about 5-40% of African ethnicity (or African American), about 5-40% of European descent (“white”), and about 5-40% of Asian ethnicity, e.g., of one or more of Chinese, Korean, Japanese, and southeast Asian ethnicity. Also, “heterogenous population” can refer to a population that is, about 5-80% of African ethnicity (or African American), about 5-80% of European ethnicity (“white”), and about 5-80% of Asian ethnicity, e.g., of one or more of Chinese, Korean, Japanese, and southeast Asian ethnicity.

“Heterogenous population” can also refer to a population that is, about 10-70% of African ethnicity (or African American), about 10-70% of European ethnicity (“white”), and about 10-70% of Asian ethnicity, e.g., of one or more of Chinese, Korean, Japanese, and southeast Asian ethnicity. The present disclosure can exclude systems and methods that involve a population of human subjects that does not fit into one of the above descriptions of “heterogenous population.”

“Heterogeneous population” can refer to a population of human subjects that is at least 5% diagnosed with diabetes mellitus, at least 5% diagnosed with hypertension, at least 5% recent smokers, and at least 5% obese. Obesity can be determined by anthropometry or Body Mass Index (BMI) (see, e.g., Brody (1999) Obesity in Nutritional Biochemistry, Academic Press, San Diego, pp. 379-419). “Heterogeneous population” can refer to a population of human subjects that is at least 10% diagnosed with diabetes mellitus, at least 10% diagnosed with hypertension, at least 10% recent smokers, and at least 10% obese. Each of these percentages can be independently varied to be, e.g., at least 5%, at least 10%, at least 15%, at least 20%, at least 25%, and such, for defining a “heterogeneous population.”

Clinical studies that use a heterogeneous population have an increased ability to detect any subgroup of interest. In contrast, clinical studies that use a homogeneous population risk failing to detect parameters that are mainly exhibited by subgroups that are not encompassed by that particular homogeneous population. Guidance on the use of subgroups for establishing a connection between the efficacy and a specific demographic population is available (see, e.g., Brody, T. (2012) Clinical Trials: study design; endpoints & biomarkers; drug safety; FDA & ICH guidelines. Elsevier, Inc., New York, N.Y., pages 65-72, 82-89).

Study Population (see, 3921 Cheng, Y. C. 2011)

Table 11 discloses the demographics of the study population.

TABLE 11
Population characteristics by case-control status

| | | Control | |
|---|---|---|---|
| Characteristic | Case (n = 889) | (n = 927) | P value§ |
| Age (mean +/− SD, | 41.3 +/− 6.9 | 39.6 +/− 6.8 | <0.001 |
| years) | | | |
| Female (%) | 41.5 | 43.6 | 0.37 |
| Self-reported ethnicity | | | |
| White | 52.42 | 56.42 | 0.22 |
| African- | 42.41 | 38.51 | |
| American | | | |
| Other | 5.17 | 5.07 | |
| Subtype (%) | | | |
| Cardioembolic | 20.0 | — | |
| Large artery | 7.1 | — | |
| Lacunar | 16.1 | — | |
| Other known | 6.5 | — | |
| cause | | | |
| Undetermined | 50.3 | — | |
| cause | | | |
| Hypertension (%) | 42.7 | 19.2 | <0.001 |
| Diabetes mellitus (%) | 16.7 | 5.1 | <0.001 |
| Angina/myocardial | 5.3 | 0.7 | <0.001 |
| infarction (%) | | | |
| Current smoker (%) | 42.5 | 28.6 | <0.001 |

§Unadjusted P values for age, sex, and race. Age and sex-adjusted P values for other characteristics.

The Genetics of Early Onset Stroke (GEOS) Study is a population based case-control study designed to identify genes associated with early-onset ischemic stroke and to characterize interactions of identified stroke genes and/or SNPs with environmental risk factors. Participants were recruited from the greater Baltimore-Washington area in four different periods: Stroke Prevention in Young Women-1 (SPYW-1) conducted from 1992 to 1996, Stroke Prevention in Young Women-2 (SPYW-2) conducted from 2001 to 2003, Stroke Prevention in Young Men (SPYM) conducted from 2003 to 2007, and Stroke Prevention in Young Adults (SPYA) conducted in 2008 (3923 Kittner, S. J. 1998). From these samples, a total of 921 cases and 941 controls that consented to having their DNA used for genetic studies of stroke. The racial and sexual mixes are disclosed herein. The age of patients in the dataset is between 15-49 years of age. The original datasets were constructed with the consent of all study participants and was approved by the University of Maryland at Baltimore Institutional Review Board (3920 Cheng, Y. C. 2011).

A total of 1814 GEOS study participants, including 889 cases and 927 controls, comprise the dataset which has been used by (3920 Cheng, Y. C. 2011) (new cole) and in our analysis. Characteristics of study participants are disclosed herein. The mean age was 41.3 years for cases and 39.6 years for controls (P<0.001). The population is primarily composed of two self-reported race groups, white (54.5%) and African American (40.4%), with the remaining 5.1% of individuals comprising other races, including Chinese, Japanese, other Asians, and other unspecified. There were more males than females among both cases and controls. Cases were more likely than controls to report having prevalent hypertension, diabetes, and myocardial infarction and to being current smokers. The publically available dataset was provided to us after application and review and included age ranges but no specific ages, sex, and race, but did not include medical information, i.e. hypertension, diabetes, myocardial infarction, or data on stroke subtype.

Further details of Plink analysis are disclosed below in Table 12.

TABLE 12A
Details of Plink analysis

```
Full Model/Association testing
It is possible to perform tests of association between a disease and a
variant other than the basic allelic test (which compares frequencies of
alleles in cases versus controls), by using the -- model option. The
tests offered here are (in addition to the basic allelic test):
    Cochran-Armitage trend test
    Genotypic (2 df) test
    Dominant gene action (1df) test
    Recessive gene action (1df) test
One advantage of the Cochran-Armitage test is that it does not assume
Hardy-Weinberg equilibrium, as the individual, not the allele, is the unit
of analysis (although the permutation-based empirical p-values from
the basic allelic test also have this property). It is important to remember
that SNPs showing severe deviations from Hardy-Weinberg are often
likely to be bad SNPs, or reflect stratification in the sample, however,
and so are probably best excluded in many cases.
The genotypic test provides a general test of association in the 2-by-3
table of disease-by-genotype. The dominant and recessive models
are tests for the minor allele (which is the minor allele can be
found in the output of either the --assoc or the --freq commands.
That is, if D is the minor allele
(and d is the major allele):
```

| | | | | | |
|---|---|---|---|---|---|
| Allelic: | D | versus | d | | |
| Dominant: | (DD, Dd) | versus | dd | | |
| Recessive: | DD | versus | (Dd, dd) | | |
| Genotypic: | DD | versus | Dd | versus | dd |

```
As mentioned above, these tests are generated with option:
  plink --file mydata --model
which generates a file
    plink.model
which contains the following fields:
```

| | |
|---|---|
| CHR | Chromosome number |
| SNP | SNP identifier |
| TEST | Type of test |
| AFF | Genotypes/alleles in cases |
| UNAFF | Genotypes/alleles in controls |
| CHISQ | Chi-squated statistic |
| DF | Degrees of freedom for test |
| P | Asymptotic p-value |

TABLE 12B
Continued details of Plink analysis

```
Each SNP will feature on five rows of the output, correspondnig to the five tests applied. The
column TEST refers to eitherALLELIC, TREND, GENO, DOM or REC, refering to the different types of
test mentioned above. The genotypic or allelic counts are given for cases and controls separately.
For recessive and dominant tests, the counts represent the genotypes, with two of the classes pooled.
These tests only consider diploid genotypes: that is, for the X chromosome males will be excluded even from
the ALLELIC test. This way the same data are used for the five tests presented here. Note that, in contrast,
the basic association commands (--assoc and --linear, etc) include single male X chromosomes, and so
the results may differ.
  The genotypic and dominant/recessive tests will only be conducted if there is a minimum number of
  observations per cell in the 2-by-3 table: by default, if at least one of the cells has a frequency less than
  5, then we skip the alternate tests (NAis written in the results file).
The Cochran-Armitage and allelic tests are performed in all cases. This threshold can be altered with the --
cell option:
  plink --file mydata --model --cell 20
If permutation (with the --mperm or --perm options) is specified, the -model option will by default perform a
permutation test based on the most significant result of ALLELIC, DOM and REC models. That is, for each
SNP, the best original result will be compared against the best of these three tests for that SNP for every
replicate. In max(T) permutation mode, this will also be compared against the best result from all SNPs for
the EMP2 field. This procedure controls for the fact that we have selected the best out of three correlated tests
for each SNP. The output will be generated in the file
  plink.model.best.perm
or
  plink.model.best.mperm
depending on whether adaptive or max(T) permutation was used.
The behavior of the --model command can be changed by adding the --model-gen, --model-trend, --
model-dom or --model-rec flags to make the permutation use the genotypic, the Cochram-Armitage trend
test, the dominant test or the recessive test as the basis for permutation instead. In this case, one of the the
following files will be generated:
```

| | |
|---|---|
| plink.model.gen.perm | plink.model.gen.mperm |
| plink.model.trend.perm | plink.model.trend.mperm |
| plink.model.dom.perm | plink.model.dom.mperm |
| plink.model.rec.perm | plink.model.rec.mperm |

It is also possible to add the --fisher flag to obtain exact p-values:
./plink --bfile mydata --model --fisher
in which case the CHISQ field does not appear. Note that the genotypic, allelic, dominant and recessive
models use the Fisher's exact; the trend-test does not and will give the same p-value as without the --
fisher flag. Also, by default, when --fisher is added, the --cell field is set to 0, i.e. to include all SNPs.

TABLE 12C
Additional, continued details of Plink analysis

```
Basic Association test
To perform a standard case/control association analysis, use the option:
  PLINK --FILE MYDATA --ASSOC
which generates a file
  plink.assoc
which contains the fields:
```

| | |
|---|---|
| CHR | Chromosome |
| SNP | SNP ID |
| BP | Physical position (base-pair) |
| A1 | Minor allele name (based on whole sample) |
| F_A | Frequency of this allele in cases |
| F_U | Frequency of this allele in controls |
| A2 | Major allele name |
| CHISQ | Basic allelic test chi-square (1df) |
| P | Asymptotic p-value for this test |
| OR | Estimated odds ratio (for A1, i.e. A2 is reference) |

Hint: In addition, if the optional command --ci X (where X is the desired coverage for a confidence
interval, e.g. 0.95 or 0.99) is included, then two extra fields are appended to this output:

| | |
|---|---|
| L95 | Lower bound of 95% confidence interval for odds ratio |
| U95 | Upper bound of 95% confidence interval for odds ratio |

(where 95 would change if a different value was used with the --ci option, naturally).
Adding the option
--counts
with --assoc will make PLINK report allele counts, rather than frequencies, in cases and controls.
See the next section on permutation to learn how to generate empirical p-values and use other
aspects of permutation-based testing.
See the section on multimarker tests to learn how to perform haplotype-based tests of association.
This analysis should appropriately handle X/Y chromosome SNPs automatically.

TABLE 12D
Further details of Plink analysis

In Plink analysis, the basic allele association testing is focused on association of SNPs in cases versus
controls, where cases represent early onset stroke from the Geneva dataset. Allele frequency for wild
type and variant SNPs can be derived from this analysis (Figure ). Significant p values for the 12 variant
SNPs in the 1-carbon folate pathway are represented as well as the rsxxx133, which has had significant
association in other studies. In addition to basic allele testing, Plink has a more extensive association
evaluation, model, that includes Cochran-Armitage trend test, Genotypic (2 df) test, Dominant gene action
(1df) test, and Recessive gene action (1df) test. Each of these association tests were applied with Plink to
the Geneva early stroke dataset that was selected for genes in the 1-carbon folate pathway (Figure). The
genotype testing is useful for determining the genotype and its numbers for specific variant SNPs. you
look at the “genotype” model to see how many heterozygous/homozygous calls you have in each of the
cases (AFF) and controls (UNAFF).
As an example for MTHFR rs7533315, genotype analysis shows:

| | | | | | | | |
|---|---|---|---|---|---|---|---|
| 1 | rs7533315 | T | C | GENO | 51/350/528 | 90/337/458 | 1 |

4.94 2 0.0005687
This tells you that:
A1= T is minor allele
A2 = C is major allele
AFF (cases):
51/350/528
51 TT
350 TC
528 CC
UNAFF (controls):
90/337/458
90 TT
337 CT
458 CC
As an example, for rs753315, the Lowest p-value is for REC model.

| | | | | | | | |
|---|---|---|---|---|---|---|---|
| 1 | rs7533315 | T | C | REC | 51/878 | 90/795 | 1 |

3.85 1 0.0001984
This could be interpreted that the T allele, which is in a recessive model here, has a protective role for
stroke, and you would need two alleles (90 TT homozygous controls vs. 51 TT homozygous cases) to
have stroke protection.
This type of evaluation using the different Plink association tools can be applied for analysis on all 12
statistically significant 1-carbon folate pathway genes and their variants.

TABLE 12E
Plink analysis. Plink 1113 model.

| CHR | SNP | A1 | A2 | TEST | AFF | UNAFF | CHISQ | DF | P |
|---|---|---|---|---|---|---|---|---|---|
| 1 | rs4846048 | G | A | GENO | 111/414/404 | 124/418/343 | 4.655 | 2 | 0.09753 |
| To read: | | | | | | | | | |
| Cases (AFF): | | | | | | | | | |
| GG/AG/AA | | | | | | | | | |
| 111/414/404 | | | | | | | | | |
| Control (UNAFF): | | | | | | | | | |
| GG/AG/AA | | | | | | | | | |
| 124/418/343 | | | | | | | | | |
| 1 | rs4846051 | G | A | GENO | 27/150/752 | 37/171/677 | 5.809 | 2 | 0.05478 |
| 1 | rs4846051 | G | A | TREND | 204/1654 | 245/1525 | 5.783 | 1 | 0.01618 |
| 1 | rs4846051 | G | A | ALLELIC | 204/1654 | 245/1525 | 6.848 | 1 | 0.008874 |
| 1 | rs4846051 | G | A | DOM | 177/752 | 208/677 | 5.368 | 1 | 0.02051 |
| 1 | rs4846051 | G | A | REC | 27/902 | 37/848 | 2.163 | 1 | 0.1414 |
| 1 | rs6541003 | G | A | GENO | 196/444/289 | 212/449/223 | 8.051 | 2 | 0.01785 |
| 1 | rs6541003 | G | A | TREND | 836/1022 | 873/895 | 6.905 | 1 | 0.008597 |
| 1 | rs6541003 | G | A | ALLELIC | 836/1022 | 873/895 | 6.985 | 1 | 0.008219 |
| 1 | rs6541003 | G | A | DOM | 640/289 | 661/223 | 7.735 | 1 | 0.005417 |
| 1 | rs6541003 | G | A | REC | 196/733 | 212/672 | 2.16 | 1 | 0.1416 |
| 1 | rs1801133 | T | C | GENO | 57/358/514 | 62/283/539 | 8.467 | 2 | 0.0145 |
| 1 | rs1801133 | T | C | TREND | 472/1386 | 407/1361 | 2.701 | 1 | 0.1003 |
| 1 | rs1801133 | T | C | ALLELIC | 472/1386 | 407/1361 | 2.802 | 1 | 0.09415 |
| 1 | rs1801133 | T | C | DOM | 415/514 | 345/539 | 5.928 | 1 | 0.01491 |
| 1 | rs1801133 | T | C | REC | 57/872 | 62/822 | 0.5693 | 1 | 0.4505 |
| 1 | rs4846052 | C | T | GENO | 217/404/308 | 169/357/359 | 11.71 | 2 | 0.002864 |
| 1 | rs4846052 | C | T | TREND | 838/1020 | 695/1075 | 11.1 | 1 | 0.000864 |
| 1 | rs4846052 | C | T | ALLELIC | 838/1020 | 695/1075 | 12.66 | 1 | 0.0003744 |
| 1 | rs4846052 | C | T | DOM | 621/308 | 526/359 | 10.71 | 1 | 0.001067 |
| 1 | rs4846052 | C | T | REC | 217/712 | 169/716 | 4.916 | 1 | 0.02661 |
| 1 | rs7533315 | T | C | GENO | 51/350/528 | 90/337/458 | 14.94 | 2 | 0.0005687 |
| 1 | rs7533315 | T | C | TREND | 452/1406 | 517/1253 | 10.69 | 1 | 0.001079 |
| 1 | rs7533315 | T | C | ALLELIC | 452/1406 | 517/1253 | 11.04 | 1 | 0.0008937 |
| 1 | rs7533315 | T | C | DOM | 401/528 | 427/458 | 4.722 | 1 | 0.02979 |
| 1 | rs7533315 | T | C | REC | 51/878 | 90/795 | 13.85 | 1 | 0.0001984 |
| 2 | SNP2-228286391 | T | C | GENO | 196/359/371 | 212/369/304 | 6.49 | 2 | 0.03896 |
| 2 | SNP2-228286391 | T | C | TREND | 751/1101 | 793/977 | 5.677 | 1 | 0.01718 |
| 2 | SNP2-228286391 | T | C | ALLELIC | 751/1101 | 793/977 | 6.689 | 1 | 0.009701 |
| 2 | SNP2-228286391 | T | C | DOM | 555/371 | 581/304 | 6.32 | 1 | 0.01194 |
| 2 | SNP2-228286391 | T | C | REC | 196/730 | 212/673 | 2.016 | 1 | 0.1556 |

TABLE 12F
Plink 113 model (continued)

| | | | | | | | | | |
|---|---|---|---|---|---|---|---|---|---|
| 5 | rs1802059 | A | G | GENO | 102/401/426 | 93/332/460 | 7.152 | 2 | 0.02798 |
| 5 | rs1802059 | A | G | TREND | 605/1253 | 518/1252 | 4.37 | 1 | 0.03658 |
| 5 | rs1802059 | A | G | ALLELIC | 605/1253 | 518/1252 | 4.609 | 1 | 0.03181 |
| 5 | rs1802059 | A | G | DOM | 503/426 | 425/460 | 6.798 | 1 | 0.009128 |
| 5 | rs1802059 | A | G | REC | 102/827 | 93/792 | 0.1048 | 1 | 0.7461 |
| 5 | rs6893970 | A | G | GENO | 8/188/733 | 10/130/745 | 9.837 | 2 | 0.007311 |
| 5 | rs6893970 | A | G | TREND | 204/1654 | 150/1620 | 6.43 | 1 | 0.01122 |
| 5 | rs6893970 | A | G | ALLELIC | 204/1654 | 150/1620 | 6.46 | 1 | 0.01104 |
| 5 | rs6893970 | A | G | DOM | 196/733 | 140/745 | 8.368 | 1 | 0.003818 |
| 5 | rs6893970 | A | G | REC | 8/921 | 10/875 | 0.3333 | 1 | 0.5637 |
| 18 | | | | | | | | | |
| 18 | rs2244500 | T | C | GENO | 185/434/309 | 161/376/348 | 7.117 | 2 | 0.02848 |
| 18 | rs2244500 | T | C | TREND | 804/1052 | 698/1072 | 5.218 | 1 | 0.02235 |
| 18 | rs2244500 | T | C | ALLELIC | 804/1052 | 698/1072 | 5.632 | 1 | 0.01763 |
| 18 | rs2244500 | T | C | DOM | 619/309 | 537/348 | 7.116 | 1 | 0.007641 |
| 18 | rs2244500 | T | C | REC | 185/743 | 161/724 | 0.8915 | 1 | 0.3451 |
| 18 | rs1001761 | C | T | GENO | 184/437/308 | 161/379/345 | 6.689 | 2 | 0.03528 |
| 18 | rs1001761 | C | T | TREND | 805/1053 | 701/1069 | 4.817 | 1 | 0.02819 |
| 18 | rs1001761 | C | T | ALLELIC | 805/1053 | 701/1069 | 5.171 | 1 | 0.02297 |
| 18 | rs1001761 | C | T | DOM | 621/308 | 540/345 | 6.684 | 1 | 0.009726 |
| 18 | rs1001761 | C | T | REC | 184/745 | 161/724 | 0.7667 | 1 | 0.3812 |
| 18 | rs2847149 | G | A | GENO | 184/437/307 | 161/379/345 | 6.855 | 2 | 0.03247 |
| 18 | rs2847149 | G | A | TREND | 805/1051 | 701/1069 | 4.936 | 1 | 0.0263 |
| 18 | rs2847149 | G | A | ALLELIC | 805/1051 | 701/1069 | 5.298 | 1 | 0.02135 |
| 18 | rs2847149 | G | A | DOM | 621/307 | 540/345 | 6.85 | 1 | 0.008865 |
| 18 | rs2847149 | G | A | REC | 184/744 | 161/724 | 0.7864 | 1 | 0.3752 |

### Definitions of Cases and Controls

“Case participants” were hospitalized with a first cerebral infarction identified by discharge surveillance from 59 hospitals in the greater Baltimore-Washington area and direct referral from regional neurologists. Ischemic stroke with the following characteristics were excluded from participation: stroke occurring as an immediate consequence of trauma; stroke within 48 hrs. after a hospital procedure, stroke within 60 days after the onset of a nontraumatic subarachnoid hemorrhage, and cerebral venous thrombosis. Additional exclusion criteria for the dataset production included:

(1) Known single-gene or mitochondrial disorders recognized by a distinctive phenotype; e.g., cerebral autosomal dominant arteriopathy with subcortical infarcts and leukoencephalopathy (CADASIL), mitochondrial encephalopathy with lactic acidosis and stroke-like episodes (MELAS), homocystinuria, Fabry disease, or sickle cell anemia;

(2) Mechanical aortic or mitral valve at the time of index stroke;

(3) Untreated or actively treated bacterial endocarditis at the time of the index stroke;

(4) Neurosyphilis or other CNS infections;

(5) Neurosarcoidosis;

(6) Severe sepsis with hypotension at the time of the index stroke;

(7) Cerebral vasculitis by angiogram and clinical criteria;

(8) Post-radiation arteriopathy;

(9) Left atrial myxoma;

(10) Major congenital heart disease;

(11) Cocaine use in the 48 hours prior to the index stroke. All cases had neuroimaging that was consistent with cerebral infarction, although neuroimaging was not used for case ascertainment.

All cases had age of first stroke between 15 and 49 years and were recruited within three years of stroke. “Control participants” without a history of stroke were identified by random-digit dialing. Controls were balanced to cases by age and region of residence in each study and were additionally balanced for race in SPYW-2 and SPYM. Traditional stroke risk factors and other study variables, including five year age range and race/ethnicity were also collected during a standardized interview. Exact age, history of hypertension diabetes, myocardial infarction (MI), and current smoking status (defined as use within one month prior to event for cases and at a comparable reference time for controls), were also collected during a standardized interview, but were not available in the dataset provided. The abstracted hospital records of cases were reviewed and adjudicated for IS subtype by a pair of neurologists according to previously published procedures (3923 Kittner, S. J. 1998) and is reducible to the more widely used TOAST system (3924 Marnane, M. 2010) that assigns each case to a single category. This IS subtype classification was not provided to us in the dataset.

### Genotyping

Genotyping from genomic DNA was performed as described (3920 Cheng, Y. C. 2011). Genomic DNA was isolated from a variety of sample types, including cell line (55.2%), whole blood (43.1%), mouth wash (0.4%), and buccal swab (0.05%). Whole-genome amplification (Qiagen REPLI-g kit, Valencia, Calif.) was used to obtain sufficient DNA for genotyping in 1.3% of samples. The distribution of sample types did not differ significantly between cases and controls (56.3% cell lines in cases vs. 54.1% in controls; 41.5% whole blood in cases vs. 44.9% in controls). Samples were genotyped at the Johns Hopkins Center for Inherited Disease Research (CIDR), and genotyping was performed using the Illumina HumanOmni1-Quad_v1-0_B BeadChip with approximately one million genome-wide SNPS (Illumina, San Diego, Calif.). Case and control samples were balanced across the plates, and self-identified whites and African Americans were placed on different plates. Samples of 50 self-reported whites were also placed on African American plates for quality control. All study samples, including 39 blind duplicates (19 whites and 20 African Americans), were plated and genotyped together with 42 HapMap control samples, including 26 Utah residents with ancestry from northern and Western Europe (CEU) and 16 Yoruba (YRI) samples, and all samples were processed together in the lab. Allele cluster definitions for each SNP were determined using Illumina BeadStudio Genotyping Module version 3.3.7, Gentrain version 1.0, and the combined intensity data from all released samples. Genotypes were not called if the quality threshold (Gencall score) was below 0.15.

Genotypes of a total of 1827 study individuals (99% of attempted samples) were released by CIDR, and all had a genotype call rate greater than 98%. Genotyping concordance rate was 99.996% based on study duplicates. A total of 1,014,719 SNPs were released by CIDR (99.83% of attempted). Genotypes were not released for SNPs that had call rates less than 85%, a cluster separation value of less than 0.2, more than 1 HapMap replicate error, more than a 5% (autosomal) or 6% (X) difference in call rate between sexes, more than 0.3% male AB frequency (X), or more than a 11.3% (autosomal) or 10% (XY) difference in AB frequency. Individual SNPs were excluded post analysis if they had excessive deviation from Hardy-Weinberg equilibrium (HWE) proportions (P<1×10⁻⁷) or genotype call rates less than 95%. Departure from HWE was assessed by chi square test among controls only and among each ethnic group separately. For this report, only SNPs having minor allele frequencies (MAF) greater than 1% and SNPs passing HWE filtering in both genetically defined European ancestry (EA) and African ancestry (AA) populations were included (N ¼ 784,766 SNPs).

### Study Design and Data Curation

The scheme for the work is demonstrated in FIGS. 4 and 5. KEGG (4022 Anonymous 2012), prior GWAS and mRNA abundance studies, and stroke biomarker data (478 Xin, X. Y. 2009; 3897 Wernimont, S. M. 2011; 3882 McNulty, H. 2012; 428 Bentley, P. 2010) (1224 Stover, P. J. 2009; 1385 Devos L, Chanson A, Liu Z, Ciappio E D, Parnell L D, Mason J B, Tucker K L, Crott J W 2008; 3916 Leclerc, D. 2007) were used to identify genes related to folate transport and sufficiency and from the 1 carbon folate dependent pathway, that was not all inclusive. Five genes from the 1 carbon folate dependent pathway, including MTHFR, FOLR1, FOLR2, MTHD1, and MTR were initially chosen for analysis. After initial analysis, this group of genes was expanded to twenty four genes from folate receptors and the folate dependent 1 carbon pathway were identified and defined using Gen Bank (4024 Anonymous 2012) and UCSC genomic browser, Build 18 (4023 Anonymous 2012) based on their chromosomal localization and the location coordinates within the specific chromosome. The genes included: MTHFR, MTRR, BHMT I and II, SHMT 1 and 2, MTR, MTRR, TYMS, FPGS, SC19A1, SC19A2, SC19A3, FOLH1, PFCT, CBS, DHFR, MTHFD1, MTHFD2, NOS3, GGH, DHFR I, FOLR1, FOLR2,

The GENEVA stroke data set tped file and tfam file had 1814 cases. The data set did not have stroke cases and controls in the tped file. The case and control phenotype data, i.e. stroke and control, and sex data was merged into the total tfam file. Exact age and race data were not available. Case controls and available other case specific data, i.e., asex, were merged with the specific tfam file from the data set with a separate sample annotation.csv file that is part of the GENEVA dataset. A PERL script was constructed that utilized data on gene name, and gene chromosomal location (chromosome, start and end positions) and was used to extract SNP and case data for the 5 and then the 24 genes from the tped file of the GENEVA stroke data set (4031 Anonymous 2012). The 5 gene and then 24 gene extracted tped file and the GENEVA dataset modified tfam file were then statistically analyzed with Plink by simple association and modeling association and by logistic regression for sex. Three groups of SNPs were identified from this data. Associations between case and control at less or equal to a p value of 0.1 (similar to prior meta-analysis studies of stroke (478 Xin, X. Y. 2009), less than or equal to 0.01, and less than or equal to 0.001. This was less conservative that traditional Bonferoni criteria. These groups were separated out from the original tped file individually by significance group and also in a combined fashion. Separate tped files from these statistical significance groups that showed significance based on case control from the PLINK analysis were then converted into CSV format files. This was done used a separately constructed C++ program followed by manipulation in Excel to produce the CSV format files for Weka data mining. These files from the single locus analysis were then used for multilocus interaction analysis in Weka using various data mining algorithms such as Random Forest and Naïve Bayes classification (4019 Frank, E. 2004). Classification accuracy of case/control status was estimated using 10-fold cross validation.

The process was repeated from extraction of the 24 gene tped file for sex, race, age, and intercurrent medical condition. The tfam file was modified to include this data. Single locus analysis with PLINK was performed and statistically significant groups as defined above were identified, used to construct a new tped file, C++, converted to CSV format, and then used for multilocus analysis in Weka. In the “Extraction phase”, the data related to 22 genes is extracted from the tped file from GENEVA STROKE dataset. There are 1814 samples in both tped and tfam files. In the “preprocessing phase”, the extracted data are processed to get base-pair information. The processed data is then transposed and used for the analysis in PLINK. Significant genes (p<0.1) are again extracted out from the processed data and used for WEKA analysis.

gPlink (1271 Purcell, S. 2007) and HapMap (4027 Anonymous 2012) and Haploview (4017 Anonymous 2011; 3918 Barrett, J. C. 2005) was used to evaluate the SNPs identified and was used for haplotype mapping and analysis (1365 Dinu, V. 2007). Haploviews were determined on individual selected genes and their SNPs based on the initial Plink p value selection filters. Linkage disequilibrium and block relationships between these SNPs for the individual genes and their SNPs was determined.

The location of the variant SNPs was analysed by haploview through gplink. Hapmap and association of genes. Haploview was used to represent the haplotype of the individual SNPs.

### Bioinformatic Functional Analysis

Those SNPs and their associated genes with p values on Plink analysis less than 0.01 were evaluated functionally by dividing SNPs for these genes into coding region/exonic SNPs and intronic and non-exonic SNPs based on their location on the chromosome. dbSNP (4026 Anonymous 2012) and GenBank (4024 Anonymous 2012) were used to identify the exact position of these SNPs within the gene/chromosome. For exonic SNPs, Poly Phen-2 was used to evaluate a protein functional effect of the SNP variance; the exact position and distance with regard to pathological SNPs within the gene and change or lack of change in an amino acid by the SNP was determined by analysis of the GenBank and UCSC gene structure data (4023 Anonymous 2012; 4024 Anonymous 2012). For non-exonic SNPs, the position and distance with regard to exons, promoters, and UTR was determined. For those that were intronic, nucleotide sequence data of the full gene was used to determine the exact sites for promoters, exons, and introns (4024 Anonymous 2012) in FASTA format (4029 Anonymous 2012) (4024 Anonymous 2012). The position of our identified SNPs was evaluated with regard to defined splice sites at the exon and intron junctions. Within the gene and intron and exons of relevance, SplicePort (3966 Dogan, R. I. 2007; 3966 Dogan, R. I. 2007) was used to identify alternative splice sites and the position of these was then related to relevant SNPs that we identified by Plink association analysis. Further the position of polypyrimidine tracts and other regulatory sequences (1373 Levy, S. 2007) and epigenetic regulatory sites for microRNAs, identified by Targetscan5.2 (4028 Anonymous 2012) and methylation patterns by Gen Bank (4024 Anonymous 2012) was related to the SNPs that we selected from the 1 carbon pathway genes. Putative binding sites for specific transcriptional regulators were also related to the site of the SNP variance (4030 Anonymous 2012). Exons, ESEs, ESS, and triplicate G sequences were determined by exon for individual Plink selected genes with ExonScan (4025 Anonymous 2012). In addition to alternative splicing sites for alternative transcripts, identified naturally occurring mutations and their chromosomal position in the identified genes, including those with reduced protein activity and amount, lack of protein production, nonfunctional proteins, splicing abnormalities, insertions, and deletions were related by position to the specific SNPs.

### Extraction of Genomic DNA and Genetic Analysis

Genomic DNA can be extracted using the QIAamp DNA Mini Kit (Qiagen, Valencia, Calif.). Single polynucleotide polymorphisms can be determined using the Taqman® fluorogenic nuclease assay (Applied Biosystems, Foster City, Calif.) and Taqman PCR Core Reagent Kit (Applied Biosystems, Foster City, Calif.). Genotype analysis can be conducted using ABI PRISM® 7900HT sequence-detection system (Applied Biosystems, Foster City, Calif., USA) using real-time PCR and TaqMan® reagents. Also, genotyping can conducted using real-time PCR analysis on the LightCycler™ (Roche Molecular Biochemicals, Indianapolis, Ind.).

### Antagonists of Blood Clotting; Antagonists of Platelet Function

Blood clotting antagonists include Warfarin®, dicoumarol, heparin, urokinase, streptokinase, tissue plasminogen activator (tPA), dypridamole. Blood clotting antagonists include inhibitors of P2Y₁₂, an adenosine diphosphate (ADP) chemoreceptor on platelet cell membranes, for example, clopidogrel.

### Stroke Subtypes

The present disclosure, in alternate embodiments, provides an assessment of risk for stroke or for one or more subtypes of stroke. The present disclosure provides an assessment of risk for stroke that arises from, or that is classified as, large-artery stenosis, small-vessel disease, and cardioembolism (Markus (2012) BMC Medicine. 10:113 (9 pages)). Stroke subtypes include cardioembolic stroke, and non-cardioembolic stroke. Stroke subtypes include, (1) Large artery atherosclerosis including patients with clinical and brain imaging findings of either significant (>50%) stenosis or occlusion of a major brain artery or cortical artery branch, probably due to atherosclerosis; (2) Cardioembolism (patients with cerebral infarction probably due to an embolus originated in the heart. Possible large artery atherosclerotic sources of thrombosis or embolism should be excluded); (3) Small artery occlusion (lacuna) including patients with classical lacunar syndromes and no evidence of cardiac embolism and significant large artery stenosis; (4) Rare causes of stroke such as nonatherosclerotic vasculopathies, hypercoagulable states, or hematologic disorders. See, e.g., Shin et al (2013) J. Cerebrovascular and Endovascular Neurosurgery. 15:131-136; Adams et al (1993) Stroke. 24:35-41; Leira et al (2008) Cerebrovasc. Dis. 26:573-577, both of which cite the TOAST criteria.

### Discussion

The identification of genes or groups of genes that increase stroke susceptibility has been limited by the small number of genes that have discrete mendelian inheritance and by the methodological and technical approaches to gene identification and the complex genetics of stroke and its subtypes as well as those of risk factors, including but not limited to atrial fibrillation, hypertension, and smoking. “The genetic variants for stroke described to date account for only a small proportion of overall stroke risk” but novel treatments can come from low predictive value SNPs in disease (3825 Markus, H. S. 2012).

The use of datasets from early onset stroke narrows the contribution of potential risk factors from lifestyle and other medical conditions and has been useful in identification by GWAS of some important genes (3853 Cole, J. W. 2011). The functional importance of these genes in the pathophysiology of stroke is less clear. In the current study, we have utilized a quality dataset of early onset stroke that has been confirmed by strict quality control analysis and developed using clear exclusions (3920 Cheng, Y. C. 2011). The analysis of this dataset by candidate gene analysis of case controls and GWAS with and without combination with other early onset stroke datasets (3853 Cole, J. W. 2011) has identified 1-carbon folate dependent pathway gene polymorphisms for 5 of 24 pathway genes, i.e. MTHFR (4), TYMS (3), MTRR (1), BHMT (1), FOLR2 (beta) and SLC19A3 (2) that modestly increase stroke risk with p values less than 0.001, and the odds ratios, as seen in other studies have remained between 1.0 and 1.5. We have approached analysis of this dataset using a candidate gene approach of genes in an important functional pathway, the folate 1 carbon pathway, extraction of the data related to these genes, and a combination of Plink association analysis with identification of the selected genes above and Weka analysis and functional bioinformatic analysis. This has been used to identify genes from this pathway that have significant associations, to evaluate relationships between these genes and stroke and the specific pathways and functions of the gene products of these genes, and to see if these specific genes and their polymorphisms have functions that contribute to novel and biologically relevant biological pathophysiologies for stroke.

The modest statistical associations in this study must be considered. The current study has limitations related to the use of GWAS in general and our results with below Bonferoni criteria p values, low Ors, the use of a stroke population without subtype analysis, and the use of total stroke population data rather than differentiated by medical condition, age, or race. The public dataset available did not include medical condition, age, race, or stroke subtype, but including these in the dataset may have not allowed sufficient power related to numbers. However, Consistent observations across multiple statistical measures with Plink were seen with the MTHFR related SNPs. Further, the fact that we have observed SNPs in the general population data set may reflect on specific DNA polymorphisms that predispose to general stroke risk. The fact that multiple genes are found in a set of related pathways may be relevant for identifying stroke risk. The low level of statistical association is consistent with multiple previous studies of stroke risk with GWAS and meta-analysis of GWAS studies. This emphasizes the difficulty in identifying stroke risk genes in a polygenic disorder with lifestyle and epigenetic contributions and where additional risk factors, i.e. hypertension, atrial fibrillation, have their own polygenic inheritance.

Current studies in process and recent studies to increase power have utilized combined data from multiple studies (3853 Cole, J. W. 2011) and these have now involved, including a non-public enhanced dataset (from the public one that we used) dataset with a redetermination of cases and controls and phenotypes with new standardized NIH software. Further, the dataset that we utilized employed an Illumina chipset with 1 million common SNPs, which limits the ability to find rare variants that may be significant. Nevertheless, available data from large size studies with subtype analysis, although showing new genes with greater significance, the number of genes identified is small and their functional and biological significance is not clear in the majority of cases. The observed SNPs in this study suggest that even using total population data from this restricted but early onset stroke dataset that common variants may be related to stroke risk. Multiple rare variants (3845 Cole, J. W. 2012), rare and common variants, and common variants may be involved in stroke susceptibility and pathophysiology (Jickling).

The one carbon pathways involving folate and involved with homocysteine metabolism, thymidylate synthesis, purine synthesis, and the generation of methylation intermediates for intermediary metabolism and DNA methylation are represented (FIGS. 1,2, and 3), The DNA polymorphisms identified are reflective of genes that we have identified are important functionally in the 1-carbon folate dependent pathway. MTHFR and MTR are directly involved with the remethylation of homocysteine to methionine and the generation of methyl intermediates. MTHFR individually is associated with stroke and MTHFR and MTR together have been associated with stroke. The MTHFR DNA polymorphisms that have been identified do not include the C677T polymorphism that has been associated with stroke, elevated homocysteine, altered folate levels, and distribution, and increased DNA uracil content as well as other pathologies; the identified MTHFR SNPs do not include SNPs that have been evaluated for disease or biochemical markers. The same is true for BHMT, MTR, TYMS, FOLR2 and SLC19A3. This suggests that these may be unique SNPs that warrant further study for stroke risk and pathophysiology.

TYMS is responsible for the conversion of uracil to thymidine, a major component of DNA. This reaction competes for methyl groups with MTHFR mediated homocysteine remethylation and other pathways that require methylation. Any alteration in the efficacy of TYMS is disadvantageous in this methyl group competition and may lead to further uracil incorporation into DNA, that may increase cancer risk. BHMT may generate up to 50% of methylation capacity with choline as the contributor. If MTHFR has reduced function, then BHMT may increase its contribution within the cell or tissue with resultant depletion of choline, which is essential for neurotransmission and myelination. Disruption of the primary critical enzymes. Alone or in combination in the 1-carbon folate dependent pathway would be predicted to be disruptive and predispose to disease pathology. However, there is currently no data available on the protein level or enzyme activity for the BHMT, MTR, or TYMs SNPs or for the MTHFR SNPs that we have identified, unlike C677T and A1289C (rs110831), which have reduced MTHFR activity. The location of the primary SNPs that we identified or within introns or in the case of MTHFR rs4846051, which is contiguous to rs110831) and BHMT, within exons as synonymous SNPs, that would not alter the base protein sequence or protein activity. None of the SNPs that we have identified are in the 5′ flanking region, which would be a site for transcriptional binding by transcriptional co-regulators nor are any in the 3′ UTR, which might site for microRNA regulation, that has been observed for MTHFR (stone). The SNPs identified are also not in sites that would influence gene methylation or suppression of gene expression. On the surface, based on this preliminary analysis, it would be difficult to attribute any functional significance to the current SNPs.

However, detailed analysis of SNP chromosomal position within the individual genes that were identified by Plink statistical analysis, shows that these locations within the gene may have important functions in splicing and alternative splicing of pre-mRNAs that may be altered by some of the specific SNPs, i.e. MTHFR rs4846052, MTHFR rs753315, MTHFR rs5846051, BHMT rs6893970, MTRR rs1802059 and SLC19A3 rs13007334. The chromosomal location for rs753315 is normally a Fox 1 and 2 putative intron splicing enhancer (ISE) that is contiguous with a general splicing enhancer, the GGG triplet. Similarly, the rs4846052 chromosomal location is also the site for a putative Fox 1 and 2 ISE. These consensus Fox 1 and 2 binding sites are removed with the SNP variances with rs7533315 in introns 2 and 3, respectively. The function of these sites is to promote alternative splicing of the pre-mRNA of MTHFR that is part of the multiple alternative transcripts that produce MTHFR functional proteins, that may be tissue and cell specific. These alterations in the Fox 1 and 2 ISE may compromise normal alternative splicing of pre-mRNA as well as the fidelity and accuracy of splicing, in general at the splice donor and acceptor sites at the exon and intron boundaries.

On the other hand, the chromosomal location for MTHFR rs4846051 is at a site without an exon splicing enhancer. The MTHFR rs4846051 creates a strong consensus exon splicing enhancer and would lead to a stimulation of alternative splicing related to this sequence. How this would affect MTHFR splicing and protein isoform production is not known. The SLC19A3 rs13007331 has GGG triplet intron splicing enhancer that is contiguous to the SNP site, but this is not altered by the polymorphism and is the same for both normal and SNP.

The MTRR rs1802059 and BHMT rs1802059 occur at exon and intron locations within the chromosomes and genes that are sites for pyramidal tract binding sites, which are exon or intron splicing silencers. Two tandem PTB sites that are continuous are seen with CUCU motif and a UCUU motif in both normal MTRR and BHMT. The MTRR and BHMT SNPs remove the CUCU motif but add an additional uridine. This potentially can alter the intron and exon splicing silencing activity with alteration of alternative splicing and protein isoform production. These observations with these important genes that were identified by Plink statistical analysis for stroke versus control patients may suggest that normally, as with other genes, that splicing and alternative splicing may be regulated by consensus exon and intron splicing enhancers and silencers. The MTHFR, MTRR, and BHMT polymorphisms and their location and alteration of consensus sites with removal, creation, or interference may represent a mechanism by which splicing and alternative splicing is altered with abnormal production or patterns of tissue and cellular isoform expression. Whether these alterations of putative functional significance influence stroke risk and pathophysiology is not clear. Additional molecular work from a bioinformatic and molecular biological standpoint is needed to investigate and confirm this new model for pre-mRNA splicing and its potential relationship to stroke.

The relevance of this potential alternative splicing regulation model for certain critical one carbon folate pathway genes is substantiated by the importance of normal splicing for MTHFR and the frequency of known MTHFR mutants that affect splicing, as well as the importance of splicing fidelity in other genes (3968 Hertel, K. J. 2008). Further, the utility of models for understanding the coordinate regulation of the one carbon folate pathway is emphasized by the computational predictive model by monte carlo in silico of microRNA regulation of the one carbon folate pathway by targeting of 3′ UTR of MTHFR, SLC19A2, MAT2A, and MTHFD2 (3977 Stone, N. 2011). MicroRNa is key mechanism that determines mRNA stability and has been implicated in disease pathology (3977 Stone, N. 2011) (Stone) including stroke (3987 Tan, J. R. 2011; 3988 Wu, P. 2012).

Using a quality dataset (GENEVA) on early onset stroke (15-49 years) and biological knowledge suggesting the 1-carbon folate dependent pathway, including MTHFR as a potential contributor to stroke risk, association analysis with multiple measures and logistic regression using Plink was done on 24 genes and their variants comparing cases with stroke and controls. 1-Carbon Pathway gene variants that reached a threshold of significance greater than 1E-02 were then evaluated with multi gene interaction within the pathway using data mining techniques such as Random forest and naïve Bayes classifiers with Weka and were then used in a functional analysis focused on splice site functioning and alternative splicing.

A combinatorial approach of biological pathway knowledge with stroke relevance combined with an early onset stroke dataset, Plink association statistics, logistic regression, and multilocus classifiers has been to identify novel common gene variants in stroke, a polygenic complex disorder and as a preliminary screen to identify genes for further evaluated of functionally and biological significance and relevance. The combination of biological pathway knowledge along with an initial screening of multiple potential pathway genes followed by additional focusing of this list of pathway genes and SNPs after statistical analysis in PLINK and subsequent use in functional analysis distinguishes this approach. Our approach has involved an integrated platform for the public dataset with a PERL extraction code for the specific genes, importation and analysis in PLINK, selection and conversion of significant gene associations with C++ and excel into a CSV file, and then integration and loading to Weka has been utilized and developed. Polyphen 2 was used to predict SNP variance sequence function along with analysis of gene structure and function with GenBank, dbSNP, BLAST Aceview, SplicePort, Exon Scan and Haploview have been employed for functional analysis with the definition of a potential novel splicing and alternative splicing model for 1 carbon folate pathway regulation.

An additional value of the approach that is that it extends prior unified platforms for approaching gene variant identification in specific disorders (1364 Dinu, V. 2007) and expands on a logistic regression and data mining approaches where common variants have been identified, i.e. Alzheimer's disease and APOE 4 (3891 Briones, N. 2012). Given the statistical significance, evaluation of relationships and models for these selected genes in Weka with multiple classifiers provides a means for further understanding of stroke risk and pathophysiology. The Weka studies were not successful in this case, because there was no single gene or set of genes of higher statistical significance and only modest statistical associations were seen for the identified stroke risk genes. However, the use of bioinformatic functional analysis with multiple tools is an additional and complimentary set of tools for understanding stroke risk and pathophysiology, both with definition of gene structure and important motifs that provide a regulatory understanding for single genes as well as groups of genes within a pathway.

The use of an integrated platform that unifies valuable open source software, promotes the use of complex datasets and that analyses data to produce potentially meaningful results in statistical and classification formats is an important approach for pathophysiology and risk studies in general and in stroke. The quality of the dataset, however, is an important consideration and future SNP platforms with both rare and common variants, as well as the use of exomic sequencing and mRNA expression dataset, and better data differentiation, including but not limited to stroke subtype and intercurrent medical condition should be utilized with our stepwise approach along with molecular studies in the future. The polygenic complexity of stroke risk and pathophysiology underlies the difficulties in identifying specific stroke genes. Candidate pathways that have critical and broad metabolic significance raise the possibility of finding significant genes or groups of genes and this will be enhanced with the approach.

### Mammalian Folate Transporters

Mammalian folate transport systems include the following. Intestinal and cellular transport of folate [5] Folate and its major metabolite, 5 methyl tetrahydrofolic acid (5 methyl THF), derived from dietary sources, enters at the intestine and through cells by the reduced folate carriers (RFC) and proton coupled folate transporter (PCFT). FR a (FOLR1) and FR β (FOLR2) are high-affinity folate-binding proteins that transport folates into cells via an endocytic mechanism. Once in the cytoplasm, the vesicle acidifies, and folate is released from the receptor [5]. SLC19A3 is a member of the SLC19 family of transporters that include the folate transporter SLC19A1, and SLC9A2. Thiamine (T [symbol] ), a cation, is transported into cells via SLC19A2 and SLC19A3. The latter receptor shows a statistical difference between stroke and control patients in the current study. The primary brain folate receptor, FR α, when dysfunctional or deficient, leads to Cerebral folate deficiency [6]. A FR β beta receptor SNP, which is statistically different in stroke versus control patients in this study, has an unknown function and has not been associated with brain [7].

MTHFR HapMap; TargetScan Analysis of miRNA that Targets MTHFR; and Exon Scan of MTHFR

The MTHFR gene has a number of sites that are sensitive to restriction enzymes, and a number of SNPs, including rs7555315 and rs4846052. MTHFR HapMap can be created, showing MTHFR gene with associated exons and SNPs, and identifying three blocks for the MTHFR gene. Block 1 contains rs4846052, rs6541003, as well as the functionally abnormal SNPs, rs1801131 and rs1801133 (FIG. 8B, FIG. 8C). In Block 2 rs753315 is very small block. Rs4846051 is not identified in any block and none of SNPs identified as having a p value less than 0.01. TargetScan6 analysis can be conducted for MiRNA targeting of MTHFR. This analysis can reveal the 3′ UTR binding sites for MiR-22 and other MiRs, as well as the sites for conserved and poorly conserved binding.

### Background Information on Gene Structure

The present disclosure provides Log Plots of Statistical significance for 1-Carbon Folate Pathway SNPs. p less 0.1, 231 SNPs were identified. Provided is specific SNP by chromosome with p values less 0.01. Chromosome 1 SNP (MTHFR), Chromosome 2 (SLC19A3), Chromosome 5(MTRR, BHMT), Chromosome 18 (TYMS). When multiple values less than 0.01 were identified by the different statistical tests, then the lowest p value was chosen.

Also provided is MTHFR gene structure, mRNAs, tissue expression, DNAase sensitivity sites, SNP positions within the gene, and rs7555315, and rs4846052. The MTHFR gene is 20,039 base pairs with 11 exons ranging in size from 102 bp to 432 bp. A primary mRNA is produced that is 7105 base pairs and the protein has a 5′ catalytic domain of 40 kd and a 3′ regulatory domain of 37 kd. The C667T SNP polymorphism is in exon 5 and converts ALA222Valine. The gene also contains a A1298C SNP GLU429ALA in exon 8. Both C677T and A1298C produce a thermolabile MTHFR with reduced activity at 55% of normal. The transcription of MTHFR is complex and alternative splicing is present. The gene contains 19 different gt-ag introns that are splicing signals. Transcription produces 15 different mRNAs, 11 alternatively spliced variants and 4 unspliced forms. There are 5 probable alternative promoters, 3 non overlapping alternative last exons and 4 validated alternative polyadenylation sites. The mRNAs appear to differ by truncation of the 5′ end, truncation of the 3′ end, presence or absence of 2 cassette exons, overlapping exons with different boundaries, alternative splicing or retention of 2 introns. 10 spliced and 3 unspliced mRNAs putatively encode functional proteins, altogether 12 different isoforms (7 complete, 1 COOH complete, 4 partial). 2 mRNA variants (1 spliced, 1 unspliced; 1 partial) do not encode functional proteins. 1425 bp of this gene are antisense to spliced gene C1orf167, 403 to CLCN6, raising the possibility of regulated alternate expression. Transcription is controlled by a complex interchange of multiple transcription initiation sites and also multiple splice donor and acceptor sites in exon 1. The regulatory region and transcriptional control of the gene is poorly understood. The gene is contiguous to the CLCN6 chloride channel gene and there is 3′ overlap for the MTHFR and CLCN6 gene; joint regulation of MTHFR and CLCN6 cannot be ruled out. The sequence control elements without a TATA box are similar to MTR, MTRR, and CBS. 15 severe naturally occurring mutations have been observed, including substitutions and deletions, that may lead to splicing abnormalities and production of reduced protein levels and reductions in activity model). 41 familial mutations with accompanying reductions in activity and homocystinemia have been noted and these are associated with peripheral neuropathy, seizures, thrombotic disorders, developmental delay, and severe homocystinemia. Stroke has been observed in two siblings with a homozygous C677T polymorphism along with a reduced folate carrier mutation G80A-RFC1. 135 naturally occurring polymorphisms (UCSC Genome Browser) have been identified in the MTHFR gene which includes 11 non synonymous SNPs. 45 common haplotypes are seen with ethnic group variation. Blacks and Caucasians have differing Hap maps. Changes in protein levels and enzyme activity, whether with common C6778T or A1298C are the primary mechanism by which non synonymous SNPs in MTHFR alter function [17]. Non synonymous SNPs leading to amino acid substitutions were predominantly located in evolutionarily conserved residues across species [17], but since the structure has not been solved, the mechanism of functional interference is not known.

The skilled artisan has information that illustrates MTHFR gene structure and haplotype MAP. The MTHFR gene with associated exons and SNPs is observed. On the LD map, three blocks are identified for the MTHFR gene. Block 1 is a larger block and contains rs4846052, rs6541003, as well as the functionally abnormal SNPs, rs1801131 and rs1801133. Although rs4846052 is contiguous to rs1801131, the HapMap data suggests that this gene may be inherited with this SNP and the rs1801131. In Block 2, rs753315 is observed and this is very small block. Rs4846051 is not identified in any block and none of SNPs identified as having a p value less than 0.01 are seen in Block 3.

The skilled artisan has information that discloses TargetScan6 analysis for MiRNA targeting of MTHFR. Information on 3′ UTR binding sites for MiR-22 and other MiRs are available to the skilled artisan, as well as the sites for conserved and poorly conserved binding.

The skilled artisan has information on exon ccan of MTHFR. The different exons for MTHFR are presented. GGG triplets are commonly seen in exons 1 and 2, where exon enhancer splicing sites (ESE) are more common as opposed to exon silencing splicing sites (ESS). This would favor alternative splicing at these sites as well as in further 3′ exons. Removal or creation of ESE and ESS sites in combination with similar intron enhancer and silencing sites may affect alternative splicing.

The skilled artisan has information that shows model of splicing and alternative splicing with 1-carbon folate dependent gene polymorphisms with p value less than 0.01.

The skilled artisan has information that discloses BHMT Gene Structure, SNPs, and Hap Map. The skilled artisan has information on location of rs683970 with regard to gene and its exons and in no Block on the Hap Map despite 3 discrete Blocks with SNPs. The skilled artisan is aware of haplotype analysis is shown and SNP location and sequence. The skilled artisan has information that shows alternative splicing of BHMT.

The skilled artisan has information that illustrates MTRR. MTRR rs1802059 gene location and HapMap. The skilled artisan has information that discloses alternative splicing. rs1802059 is located in a large block, Block 3 of 3 Blocks on the Hap Map.

The skilled artisan has information that illustrates TYMS gene structure with SNP location and locations of three SNPs, rs1142500, 1001761, and rs2847149 are all located in the single Block 1 with the associated haplotype analysis.

### Exclusionary Embodiments

The system and methods of the present disclosure can exclude any system or method that uses data from one or more of the following genes: matrix metalloproteinase 9; S100 calcium-binding proteins P, A12 and A9; coagulation factor V; arginase I; carbonic anhydrase IV; lymphocyte antigen 96 (CD96); monocarboxylic acid transporter 6; ets-2 (erythroblastosis virus E26 oncogene homolog 2); homeobox gene Hox 1.11; cytoskeleton-associated protein 4; N-formylpeptide receptor; ribonuclease-2; N-acetylneuraminate pyruvate lyase; BCL6; glycogen phosphorylase (see, e.g., Tang et al (2006) J. Cereb. Blood Flow Metab. 26:1089-1102).

Further, each of the various elements of the invention and claims may also be achieved in a variety of manners. This disclosure should be understood to encompass each such variation, be it a variation of an embodiment of any apparatus embodiment, a method or process embodiment, or even merely a variation of any element of these.

Particularly, it should be understood that as the disclosure relates to elements of the invention, the words for each element may be expressed by equivalent apparatus terms or method terms—even if only the function or result is the same.

Such equivalent, broader, or even more generic terms should be considered to be encompassed in the description of each element or action. Such terms can be substituted where desired to make explicit the implicitly broad coverage to which this invention is entitled.

It should be understood that all actions may be expressed as a means for taking that action or as an element which causes that action. Similarly, each physical element disclosed should be understood to encompass a disclosure of the action which that physical element facilitates.

Any patents, publications, or other references mentioned in this application for patent are hereby incorporated by reference.

Finally, all references listed in the Information Disclosure Statement or other information statement filed with the application are hereby appended and hereby incorporated by reference; however, as to each of the above, to the extent that such information or statements incorporated by reference might be considered inconsistent with the patenting of this/these invention(s), such statements are expressly not to be considered as made by the applicant.

In this regard it should be understood that for practical reasons and so as to avoid adding potentially hundreds of claims, the applicant has presented claims with initial dependencies only.

Support should be understood to exist to the degree required under new matter laws—including but not limited to 35 USC §132 or other such laws—to permit the addition of any of the various dependencies or other elements presented under one independent claim or concept as dependencies or elements under any other independent claim or concept.

To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, the use of the transitional phrase “comprising” is used to maintain the “open-end” claims herein, according to traditional claim interpretation. Thus, unless the context requires otherwise, it should be understood that the term “compromise” or variations such as “comprises” or “comprising”, are intended to imply the inclusion of a stated element or step or group of elements or steps but not the exclusion of any other element or step or group of elements or steps.

Such terms should be interpreted in their most expansive forms so as to afford the applicant the broadest coverage legally permissible.

It should also be understood that a variety of changes may be made without departing from the essence of the invention. Such changes are also implicitly included in the description. They still fall within the scope of this invention. It should be understood that this disclosure is intended to yield a patent covering numerous aspects of the invention both independently and as an overall system and in both method and apparatus modes.

While the system, compositions, and methods, have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

What is claimed is:

1. A method for administering a treatment to a human subject that reduces risk for early onset ischemic stroke, or a treatment to the human subject that assesses risk for early onset stroke, comprising, in combination:
detecting the status (presence or absence) of one or more single nucleotide polymorphisms (SNPs) in genes selected from:
SLC19A3 gene, methylenetetrahydrofolate reductase (MTHFR), thymidylate synthase (TYMS), methionine synthase reductase (MTRR), betaine homocysteine S-methyltransferase (BHMT), and folate receptor 2 (FOLR2),
wherein the one or more SNPs is selected from: rs7533315; rs4846052; rs6541003; rs4846051; rs1802059; rs6893970; SNP2-228286391; rs13007334; rs1001761; rs2847149; rs2244500; rs229844 (“SNP list”), wherein the SNPs were identified in a database using Early Age Onset Stroke Association Algorithm,
followed by the step of implementing a treatment that uses the detected status (presence of SNP or absence of SNP), wherein if the subject comprises the SNP, administering one or both of: (i) treatment that reduces risk for early onset stroke, or (ii) treatment that is diagnostic for assessing risk for early onset stroke.

2. The method of claim 1, further comprising the steps of withdrawing at least one cell from the human subject, and processing the at least one cell to provide a source of genomic DNA suitable for identifying single nucleotide polymorphisms (SNPs).

3. The method of claim 1, wherein the treatment that diagnoses risk for early onset stroke comprises one or more of stimulating the tissues of the subject's body to vibrate using ultrasound vibration, stimulating the excitation of hydrogen atoms in the subject's body using Magnetic Resonance Imaging (MRI), and causing ionization of organic molecules in the subject's body using computed tomography (CT).

4. The method of claim 1, wherein the treatment that reduces risk for early onset stroke comprises administering one or more of folic acid, vitamin B12, vitamin B6, thiamin, aspirin, platelet antagonist, blood clotting antagonist, HDL cholesterol reducing agent, anti-hypertriglyceridemia agent, and anti-hypertensive agent.

5. The method of claim 1, wherein the one or more SNPs does not comprise any SNP that is not the SNP list.

6. The method of claim 1, wherein the one or more SNPs comprises at least two SNPs selected from the SNP list, and that further comprises at least one SNP that is not in the SNP list.

7. The method of claim 1, wherein the one or more SNPs comprises at least two SNPs selected from the SNP list, wherein at least one of the at least two SNPs selected from the SNP list is a SNP that is homozygous in the patient's genome.

8. The method of claim 1, wherein the one or more SNPs comprises at least two SNPs selected from the SNP list, wherein at least two of the at least two SNPs selected from the SNP list is a SNP that is homozygous in the patient's genome.

9. The method of claim 1, wherein the one or more SNPs comprises at least three SNPs selected from the SNP list, wherein at least three of the at least two SNPs selected from the SNP list is a SNP that is homozygous in the patient's genome.

10. The method of claim 1, wherein the one or more SNPs comprises at least two SNPs selected from the SNP list, wherein at least one of the at least two SNPs selected from the SNP list is associated with abnormal splicing.

11. The method of claim 1, wherein the one or more SNPs comprises at least two SNPs selected from the SNP list, wherein at least two of the at least two SNPs selected from the SNP list is associated with abnormal splicing.

12. The method of claim 1, wherein the one or more SNPs comprises at least three SNPs selected from the SNP list, wherein at least three of the at least two SNPs selected from the SNP list is associated with abnormal splicing.

13. The method of claim 1, wherein the single nucleotide polymorphism is a mutation is in one or more of a coding region of the gene, a promoter of the gene, a splicing region of the gene, an enhancer of the gene, an intron, a region of the chromosome corresponding to an upstream untranslated region (UTR), a region of the chromosome corresponding to a downstream untranslated region (UTR), or a region characterized by simultaneous residence in two different genes where one gene resides in Watson strand, and the other gene resides in Crick strand.

14. The method of claim 1, further comprising assessing status for at least one vitamin concurrently with said detecting the status of one or more single nucleotide polymorphisms (SNPs) in genes.

15. The method of claim 1, further comprising assessing status for at least one vitamin concurrently with said detecting the status of one or more single nucleotide polymorphisms (SNPs) in genes, wherein the status for at least one vitamin comprises assessing status for one or more of folate, vitamin B6, vitamin B12, and thiamin.

16. The method of claim 1, wherein the method is not used for a subject that comprises hemorrhagic stroke, or not used for a subject that comprises late onset stroke.

17. The method of claim 1, wherein the one or more SNPs comprises at least two SNPs selected from the SNP list, wherein at least one of the at least two SNPs selected from the SNP list is a SNP that is heterozygous in the patient's genome.

18. The method of claim 1, wherein the one or more SNPs comprises at least two SNPs selected from the SNP list, wherein at least two of the at least two SNPs selected from the SNP list is a SNP that is heterozygous in the patient's genome.

19. A method for administering a treatment to a human subject that reduces risk for early onset stroke, or a treatment to the human subject that assesses risk for early onset stroke, comprising, in combination:
withdrawing at least one cell from the human subject, and processing the at least one cell to provide a source of genomic DNA suitable for identifying single nucleotide polymorphisms (SNPs),
detecting the status (presence or absence) of one or more single nucleotide polymorphisms (SNPs) in genes selected from:
SLC19A3 gene, methylenetetrahydrofolate reductase (MTHFR), thymidylate synthase (TYMS), methionine synthase reductase (MTRR), betaine homocysteine S-methyltransferase (BHMT), and folate receptor 2 (FOLR2),
wherein the one or more SNPs is selected from: rs7533315; rs4846052; rs6541003; rs4846051; rs1802059; rs6893970; SNP2-228286391; rs13007334; rs1001761; rs2847149; rs2244500; rs229844, wherein the SNPs were identified in a database using Early Age Onset Stroke Association Algorithm.

20. The method of claim 19, followed by the step of implementing a treatment that uses the detected status (presence of SNP or absence of SNP), wherein if the subject comprises the SNP, administering one or both of: (i) treatment that reduces risk for early onset stroke, or (ii) treatment that is diagnostic for assessing risk for early onset stroke.

21. The method of claim 21, wherein the method is not used with a subject that comprises hemorrhagic stroke.

22. A system comprising, in combination:
at least a computer that is configured to apply the Early Stroke Algorithm to identify Single Nucleotide Polymorphisms (SNPs) that are significantly associated with risk for early onset stroke, wherein the significance of the association has a P value of less than 0.05.

23. The system of claim 22, wherein the significance of the association has a P value that is less than 0.005.
